(19) United States
(12) Patent Application Publication
(10) Pub. No.: US 2019/0029811 A1
(43) Pub. Date: Jan. 31, 2019

(54) HEART VALVE

(71) Applicant: DFM, LLC, Incline Village, NY (US)

(72) Inventors: Gordon B. Bishop, Santa Rosa, CA (US); Nathan Brown, Santa Rosa, CA (US); Ken Bruner, Windsor, CA (US); Darryll Fletcher, Santa Rosa, CA (US); Sean Watkins, Calistoga, CA (US)

(21) Appl. No.: 16/072,469

(22) PCT Filed: Feb. 8, 2017
(86) PCT No.: PCT/US2017/017020

§ 371 Date: Jul. 24, 2018

Related U.S. Application Data
(60) Provisional application No. 62/294,945, filed on Feb. 12, 2016.
(60) Provisional application No. 62/413,924, filed on Oct. 27, 2016.

(51) Int. Cl. A61F 2/24

(52) U.S. Cl. CPC A61F 2/2409; A61F 2/2418; A61F 2/2436; A61F 2220/0075; A61F 2250/0003; A61F 2230/0008; A61F 2220/0008

(57) ABSTRACT

An inflatable cardiovascular prosthetic implant is provided. The implant has two inner rings that support a one-way valve that allows flow through the implant. The implant has an outer ring positioned between the two inner rings and extending radially beyond the two inner rings. The implant has anchors that attach to heart tissue to help seat the implant in the annulus of the native valve.

### INCORPORATION BY REFERENCE TO ANY PRIORITY APPLICATIONS

This application claims priority to U.S. Provisional Patent Application Ser. No. 62/294,945, filed Feb. 12, 2016 and U.S. Provisional Patent Application Ser. No. 62/413,924, filed Oct. 27, 2016, the entirety of both of these priority applications are hereby expressly incorporated by reference herein.

### BACKGROUND

### Field

The present disclosure relates to medical methods and devices, and, in certain arrangements, to methods and devices for percutaneously implanting a valve.

### Description of the Related Art

The human heart has four chambers: the right and left atria, and the right and left ventricles. The atria receive blood and pump it into the ventricles. The ventricles are more muscular than the atria and generate the pressure required to pump blood throughout the body. The right ventricle pumps blood through the pulmonary circulation to oxygenate the blood. The left ventricle pumps the oxygenated blood through the systemic circulation to supply oxygen and nutrients to the tissues of the body.

The heart has four valves that direct blood flow in the correct direction during the cardiac cycle. The valves ensure that the blood does not flow from the ventricles into the corresponding atria, or flow from the arteries into the corresponding ventricles. The mitral valve (also known as the bicuspid valve or left atrioventricular valve) lies between the left atrium and the left ventricle. The mitral valve has two leaflets. The perimeter of the leaflets is attached to a fibrous annulus, and the free edges of the leaflets are tethered to subvalvular tendinous chords and papillary muscles that extend from the left ventricle. The tendinous chords and papillary muscles prevent the valve leaflets from prolapsing into the left atrium during the contraction of the left ventricle.

Various cardiac diseases or degenerative changes may cause dysfunction in any of these portions of the mitral valve apparatus, causing the mitral valve to become abnormally narrowed or dilated, or to allow blood to leak (i.e. regurgitate) from the left ventricle back into the left atrium. Valve malfunction can result from the chords becoming stretched, and in some cases tearing. When a chord tears, the result can be a failed leaflet. Also, a normally structured valve may not function properly because of an enlargement of the valve annulus pulling the leaflets apart. This condition is referred to as a dilation of the annulus and generally results from heart muscle failure. In addition, the valve may be defective at birth or because of an acquired disease, usually infectious or inflammatory. Any such impairments compromise cardiac sufficiency, and can be debilitating or life threatening.

Numerous surgical methods and devices have been developed to treat mitral valve dysfunction, including open-heart surgical techniques for replacing, repairing or reshaping the native mitral valve apparatus, and for the surgical implantation of various prosthetic devices such as annuloplasty rings to modify the anatomy of the native mitral valve. Due to the highly invasive nature of open heart valve repair or replacement, many patients, such as elderly patients, patients having recently undergone other surgical procedures, patients with comorbid medical conditions, children, late-stage heart failure patients, and the like, are often considered too high-risk to undergo heart valve surgery and are relegated to progressive deterioration and cardiac enlargement. Often, such patients have no feasible alternative treatments for their heart valve conditions.

More recently, less invasive catheter based techniques for the delivery of replacement heart valve assemblies have been developed. In some techniques, an expandable prosthetic valve can be mounted within a catheter and advanced through a blood vessel (e.g., artery, vein) to the implantation site. The prosthetic valve can then be expanded to its functional size and anchored in place to replace the defective native valve. While these devices and methods are promising treatments for valvar insufficiency, they can be difficult to deliver, expensive to manufacture, and/or may not be indicated for all patients. Therefore, it would be desirable to provide improved devices and methods for the treatment of valvar insufficiency such as mitral insufficiency.

### SUMMARY

The systems, methods and devices described herein have innovative aspects, no single one of which is indispensable or solely responsible for their desirable attributes. Without limiting the scope of the claims, some of the advantageous features will now be summarized.

Devices, systems and methods of the present disclosure can be used to facilitate transvascular, minimally invasive and other “less invasive” surgical procedures, by facilitating the delivery of treatment devices at a treatment site. “Less invasive,” for the purposes of this application, means any procedure that is less invasive than traditional, large-incision, open surgical procedures. Thus, a less invasive procedure may be an open surgical procedure involving one or more relatively small incisions, a procedure performed via transvascular percutaneous access, a transvascular procedure via cut-down, a laparoscopic or other endoscopic procedure, or the like. Generally, any procedure in which a goal is to minimize or reduce invasiveness to the patient may be considered less invasive. Furthermore, although the terms “less invasive” and “minimally invasive” may sometimes be used interchangeably in this application, neither these nor terms used to describe a particular subset of surgical or other procedures should be interpreted to limit the scope of the disclosure. Generally, devices and methods of the disclosure may be used in performing or enhancing any suitable procedure.

The present application typically describes devices, systems and methods for performing heart valve repair procedures, and more specifically heart valve replacement procedures such as mitral valve replacement to treat mitral regurgitation or incompetence. Devices and methods of the disclosure, however, may find utility in other suitable procedures, both cardiac and non-cardiac. For example, certain features and aspects of the disclosure herein may be used in procedures to other valves of the heart or body, to repair an atrial-septal defect, to access and possibly perform a valve repair or other procedure. Therefore, although the following description typically focuses on mitral valve replacement and other heart valve repair, such description should not be interpreted to limit the scope of the disclosure.

In many cases, methods of the present disclosure will be performed on a beating heart. Access to the beating heart may be accomplished by any available technique, including intravascular, transthoracic, and the like. For example, to perform a procedure on a mitral valve, a catheter may be advanced transapically through an incision at the apex of the left ventricle, and advanced toward the left artrium of the heart, to contact a length of the mitral valve. In some arrangements, access may be gained intravascularly through the arterial or venous system. For example, transfermoral access can include gaining access to the arterial system through a femoral artery and then advancing a delivery device to the aorta, into the left ventricle and up to the mitral valve. Transaortic access can include gaining access to the arterial system through aorta and advancing a delivery device into the left ventricle and up to the mitral valve. Access through the venous system can be done using a transseptal approach in which access can be gained through a central vein, into the right atrium of the heart, and across the interatrial septum to the left side of the heart to contact a length of the mitral valve. In either of these two types of intravascular access, the catheter will often be advanced, once it enters the left side of the heart, into a space defined by the left ventricular wall, one or more mitral valve leaflets, and chordae tendineae of the left ventricle. This space can provide a conduit for further advancement of the catheter to a desired location for performing mitral valve repair. In other embodiments, a catheter device may access the coronary sinus and a valve procedure may be performed directly from the sinus. A transatrial approach can be used to perform a procedure on a mitral valve. For example, an introducer may be advanced through an incision in a wall of the left atrium, providing a port for introducing a delivery catheter into the left atrium. The delivery catheter can be advanced through the introducer sheath and into the left atrium of the heart, allowing access to the mitral valve from above. Furthermore, in addition to beating heart access, methods of the present disclosure may be used for intravascular stopped heart access as well as stopped heart open chest procedures. Any suitable intravascular or other access method is contemplated within the scope of the disclosure.

In accordance certain aspects of present disclosure, there is provided an inflatable or formed in place support for a translumenally implantable heart valve, in which a plurality of tissue supports are flexible and/or movable throughout a range in a radial direction. As used herein, a radial direction is a direction which is transverse to the longitudinal axis of the flow path through the valve.

One aspect of the present disclosure comprises a cardiovascular prosthetic valve implant. The implant comprises a cuff having an inner surface that defines a pathway for blood flow across the implant. The implant has a valve positioned within the pathway. The valve is attached to the inner surface of the cuff and is configured to permit flow in a first direction through the implant and inhibit flow in a second direction opposite to the first direction. The implant has an inflatable structure that is coupled to the cuff and includes at least an inflow ring, an outflow ring, and an atrial ring. The atrial ring has an outer diameter that is greater than the outer diameter of the inflow and outflow rings. In some aspects, the cuff of the implant extends between the inflow ring and the outflow ring. In certain aspects, the implant includes a skirt that extends between the inflow ring, the atrial ring, and the outflow ring. In some aspects, a space is defined between the skirt and the cuff. In some aspects, the skirt material permits blood to enter the space between the skirt and the cuff. In some aspects, the atrial ring has an ellipse shape. In certain aspects, the inflow ring and the outflow ring are positioned off-center relative to the atrial ring.

Another aspect of the present disclosure is a cardiovascular prosthetic valve implant that has a cuff having an inner surface that defines a pathway for blood flow. The cuff is supported by an inflatable structure that includes at least one ring. A valve is positioned within the pathway and is coupled to the cuff. The valve permits flow in a first direction through the implant and inhibits flow in a second axial direction opposite to the first direction. The implant has an atrial flange that comprises an atrial ring and a skirt that extends between the cuff and the ring of the atrial flange. In some aspects, a space is defined between the skirt and the cuff. In certain aspects, the skirt is formed from a material that permits blood to enter the space between the skirt and the cuff. In some aspects, the ring of the atrial flange has an ellipse shape. In some aspects, the ring of the cuff is positioned off-center with respect to the ring of the atrial flange.

Another aspect of the present disclosure is a cardiovascular prosthetic valve implant that has a tubular cuff having an inner surface that defines a pathway for blood flow. The tubular cuff has a first end having a first diameter and a second end having a second diameter. A valve is positioned within the pathway and is coupled to the tubular cuff. The valve is configured to permit flow in a first axial direction through the implant and to inhibit flow in a second axial direction opposite to the first axial direction. The implant has an atrial flange that comprises an atrial ring having a diameter greater than the first and second ends of the tubular cuff. A skirt extends from the first end of the tubular cuff to the atrial ring and from the atrial ring to a second end of the tubular cuff to form a space between the skirt and the tubular cuff. In some aspects, the skirt is formed by a material that permits blood to enter the space between the skirt and the cuff. In certain aspects, the atrial ring of the atrial flange has an ellipse shape. In some aspects, the tubular cuff is positioned off-center with respect to the ring of the atrial flange.

Another aspect of the present disclosure is a cardiovascular prosthetic valve implant having a flexible cuff, an inflatable structure, a valve, and at least one anchor. The flexible cuff has a distal end and a proximal end. The inflatable structure is coupled to the cuff and has at least one inflatable channel that forms a ring. The valve is mounted to the cuff and is configured to permit flow in a first direction and to inhibit flow in a second direction opposite to the first direction. The at least one anchor is moveable between a first position in which the anchor is in a straight configuration and a second position in which that anchor is in a spiral configuration.

Another aspect of the present disclosure is a method of manufacturing a cardiovascular prosthetic valve implant. The method includes providing a cardiovascular prosthetic valve implant that is configured to replace a first valve of a heart. The method includes coupling the cardiovascular prosthetic valve implant to an arterial flange having a larger outer diameter than the outer diameter of the cardiovascular prosthetic valve implant such that the cardiovascular prosthetic valve implant can be positioned within a second valve of the heart. In some aspects, the method includes adding a skirt between the arterial flange and the cardiovascular prosthetic valve implant. The skirt is formed of a material that permits blood to enter a space between the skirt and the cardiovascular prosthetic valve implant. Another aspect of the present disclosure is a cardiovascular prosthetic valve implant having a tubular cuff, a valve, and an atrial flange. The tubular cuff has an inner surface that defines a pathway for blood flow. The tubular cuff has a first end having a first diameter and a second end having a second diameter. The valve is positioned within the pathway and is coupled to the cuff. The valve permits flow in a first axial direction through the implant and inhibits flow in a second axial direction opposite to the first axial direction. The atrial flange includes an atrial ring having a diameter greater than the first and second ends of the tubular cuff. The tubular cuff is positioned off-center with respect to the atrial ring of the atrial flange. In some aspects, the atrial ring has an ellipse shape.

Another aspect of the present disclosure is a method of implanting a prosthetic valve within the heart. The method includes transapically advancing a prosthetic valve having an inflatable support structure to a position proximate of a mitral valve of the heart. The method includes advancing a distal portion of the support structure past the mitral valve. The method includes inflating a distal portion of the inflatable support structure. The method includes proximally retracting the valve to seat a distal portion of the inflatable support structure against an atrial surface of the mitral valve. The method includes grasping with an anchor positioned on a proximal end of the valve fibrotic tissue surrounding the mitral valve annulus on a ventricle side of the mitral valve.

Another aspect of the present disclosure is a method of implanting a prosthetic valve within the heart. The method includes advancing a deployment catheter including the prosthetic valve to a position proximate of the native valve of the heart. The prosthetic valve includes at least one anchor positioned in a straight configuration that extends parallel to a longitudinal axis of the deployment catheter. The method includes deploying the prosthetic valve. The method includes releasing the at least one anchor and allowing the anchor to return to a spiral configuration.

Another aspect of the present disclosure is an implant anchoring system that includes a first anchor, a second anchor, and a hoop structure that connects the first anchor to the second anchor. The first and second anchors are moveable between an extended configuration and a deployed configuration. The hoop structure receives a first torque from the first anchor when the first anchor moves from the extended configuration to the deployed configuration. The hoop structure receives a second torque from the second anchor when the second anchor moves from the extended configuration to the deployed configuration. The first torque counteracts the second torque.

Another aspect of the present disclosure is a cardiovascular prosthetic valve implant that includes a tubular cuff, a valve, and an anchor. The tubular cuff has an inner surface that defines a pathway for blood flow. The valve is positioned within the pathway and is attached to the tubular cuff. The valve includes one or more leaflets that are attached to an inner surface of the cuff. The one or more leaflets permit flow in a first axial direction through the implant and inhibit flow in a second axial direction opposite to the first axial direction. The anchor is attached to the tubular cuff and includes a bend. When the valve is viewed in the second axial direction, at least a portion of the bend extends radially inward of an inner surface of the cuff.

Another aspect of the present disclosure is a method of retrieving a prosthetic valve within the heart. The method includes advancing a prosthetic valve that has a support structure out of a deployment catheter. The method further includes partially deploying the prosthetic valve. The method further includes retrieving the prosthetic valve by retracting the prosthetic valve in a sideways orientation into the deployment catheter.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 is a cross-sectional view of a heart and its major blood vessels.

FIG. 2 is a schematic representation of the mitral valve annulus from the ventricular perspective.

FIG. 3 is a cross-sectional view of a heart showing the placement of an embodiment of the implant of the present disclosure.

FIG. 4A is a perspective view of an embodiment of an implant.

FIG. 4B is a side view of the implant of FIG. 4A.

FIG. 4C is a top view of the implant of FIG. 4A.

FIG. 4D is a side view of an embodiment of an implant seated within a mitral valve annulus.

FIG. 4E is a side view of an embodiment of an implant.

FIG. 4F is a side view of an embodiment of an implant.

FIG. 5A is a cross-sectional view of the implant of FIG. 4A.

FIG. 5B is a perspective view of an embodiment of an inflatable structure.

FIG. 5C is a cross-sectional view of an embodiment of a flow channel attached to a cuff.

FIG. 5D is a cross-sectional view of an embodiment of a connection port and PFL tube.

FIG. 6A is a top view of an embodiment of an anchor.

FIG. 6B is a side view of an anchor.

FIG. 6C is a side view of an anchor in a straight configuration.

FIG. 6D is a side view of an anchor in a spiral configuration.

FIG. 6E is a top view of an embodiment of an anchor.

FIG. 7 is a perspective view of an embodiment of an anchor.

FIG. 8A is a side view of an embodiment of an implant.

FIG. 8B is a bottom view of the implant of FIG. 8A.

FIG. 8C is a partial bottom view of the implant of FIG. 8A.

FIG. 8D is a schematic diagram showing various configurations of a first bend.

FIG. 9A is a perspective view of an embodiment of a delivery catheter with an implant stowed inside the catheter.

FIG. 9B is a close up view of the delivery catheter of FIG. 9A.

FIG. 9C is a perspective view of an embodiment of a delivery catheter with an implant deployed from inside of the catheter.

FIG. 9D is a close up view of the delivery catheter of FIG. 9C.

FIG. 10 is a cross-sectional view of a heart showing trans-apical delivery of an implant to the mitral valve annulus.

FIGS. 11A-C illustrates time sequence steps of deploying an artificial valve implant.

FIG. 12 is a schematic side view of a method of testing an implant.

FIG. 13A is a side view of an embodiment of an implant, with anchors in the extended configuration.

FIG. 13B is a side view the implant of FIG. 13A, with anchors in a partially-extended configuration.

FIG. 13C is a side view the implant of FIG. 13A, with anchors in a partially-extended configuration.

FIG. 13D is a side view the implant of FIG. 13A, with anchors in a deployed configuration.

FIG. 14 is a force curve for an anchor of the implant of FIG. 13A.

FIGS. 15A-C is a side perspective view of an embodiment of recovery catheter for retrieving the implant in the patient.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

### Overview

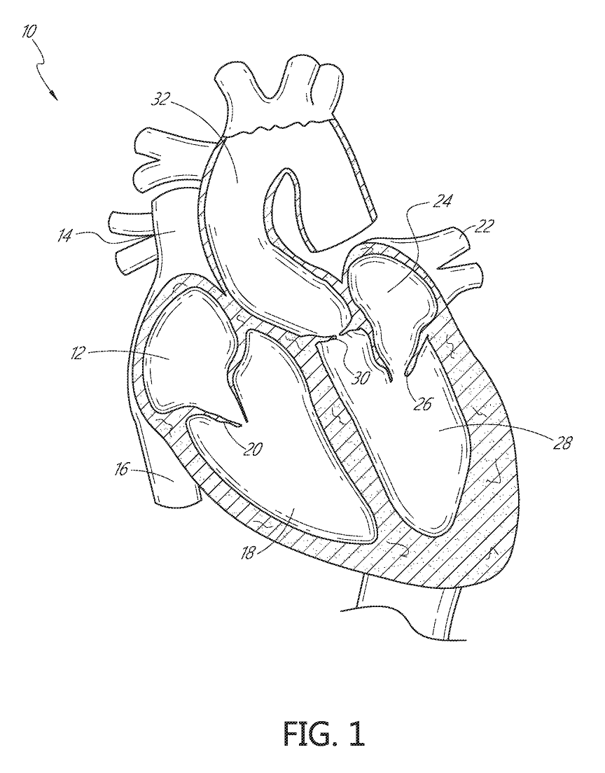

FIG. 1 is a schematic cross-sectional illustration of the anatomical structure and major blood vessels of a heart 10. Deoxygenated blood is delivered to the right atrium 12 by the superior and inferior vena cava 14, 16. Blood flows from the right atrium 12 into the right ventricle 18 through the tricuspid valve 20. Contraction of the right ventricle 18 drives this blood through the pulmonary valve (not shown) and into the pulmonary arteries (not shown). The pulmonary circulation carries the blood to the lungs for a gaseous exchange of oxygen. The circulatory pressures return the oxygenated blood back to the heart via the pulmonary veins 22 and into the left atrium 24. As the left atrium 24 fills, the mitral valve 26 opens to allow blood to be drawn into the left ventricle 28. Contraction of the left ventricle 28 expels the blood through the aortic valve 30 and in to the aorta 32. The arteries of the systemic circulation carry the blood to the capillary beds of the body tissues. The veins of the systemic circulation gather the blood from the capillary beds and return it to the right atrium 12, completing the loop of the circulatory system. When the heart 10 fails to continuously produce normal flow and pressures, a disease commonly referred to as heart failure occurs.

One cause of heart failure is failure or malfunction of one or more of the valves of the heart 10. For example, the mitral valve 26 or the aortic valve 30 can malfunction for several reasons. The mitral or aortic valve 26, 30 may be abnormal from birth or could become diseased with age. In such situations, it can be desirable to replace the abnormal or diseased valve 26, 30.

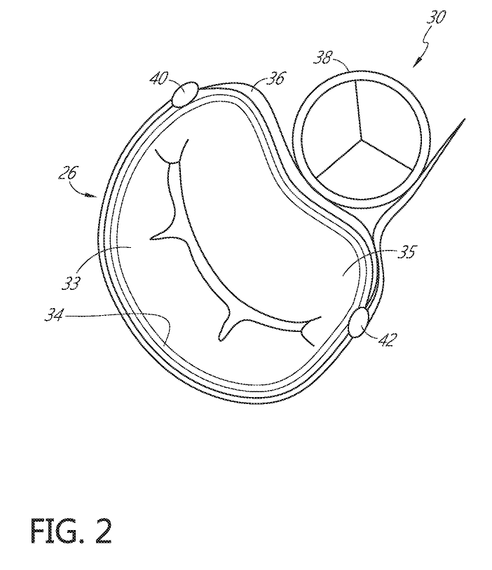

FIG. 2 depicts a schematic representation of the mitral valve 26 and the aortic valve 30 from the left ventricular perspective. The valves of the heart 10 are surrounded by fibrotic tissue that provides support to the valve. For example, the mitral valve annulus 34 is a fibrotic ring that consists of an anterior part and a posterior part and defines the opening area of the mitral valve 26. The aortic-mitral curtain 36 is a fibrous structure that connects the anterior mitral annulus 34 with the aortic valve annulus 38. The aortic-mitral curtain 36 ends at both lateral sides of the mitral valve 26 to form a left fibrous trigone 40 and right fibrous trigone 42. The left and right trigones 40, 42 are thickened areas of tissue between the aortic ring and the atrioventricular ring. The trigones 40, 42 are nearly aligned with the coaptation plane of the posterior and anterior leaflets 33, 35 of the mitral valve 26. The right fibrous trigone is between the aortic ring and the right atrioventricular ring. The left fibrous trigone is between the aortic ring and the left atrioventricular ring. As discussed in more detail below, the implant of the present disclosure can include one or more features that grasp, pierce, or otherwise attach to the fibrous tissue surrounding the valve annulus, thereby helping to secure the implant in the valve annulus.

In the description below, the present disclosure will be described primarily in the context of replacing or repairing an abnormal or diseased mitral valve 26. However, various features and aspects of methods and structures disclosed herein are applicable to replacing or repairing the aortic 26, the pulmonary, and/or the tricuspid 20 valves of the heart 10, as those of skill in the art will appreciate in light of the disclosure herein. In addition, those of skill in the art will also recognize that various features and aspects of the methods and structures disclosed herein can be used in other parts of the body that include valves or can benefit from the addition of a valve, such as, for example, the esophagus, stomach, ureter and/or vesicle, biliary ducts, the lymphatic system and in the intestines.

In addition, various components of the implant and its delivery system will be described with reference to a coordinate system comprising “distal” and “proximal” directions. In this application, distal and proximal directions refer to the perspective of the person operating a deployment system 200 (e.g., delivery catheter 200) that is used to deliver the implant 100. Thus, in general, proximal means closer to the person operating the deployment system 200 while distal means further from the person operating the deployment system 200. In addition, the terms “inflow” and “outflow” may also be used with reference to the coordinate system of the implant. In general, inflow and outflow directions refer to the perspective of normal blood flow through the circulatory system, as described above. Thus, the inflow portion of an implant 100 seated in the annulus of the mitral valve 26 would face the left atrium 24 because in normal blood flow, blood flows from the left atrium 24 to the left ventricle 28. In other words, inflow refers to the upstream direction of normal blood flow while outflow refers to the downstream direction of normal blood flow.

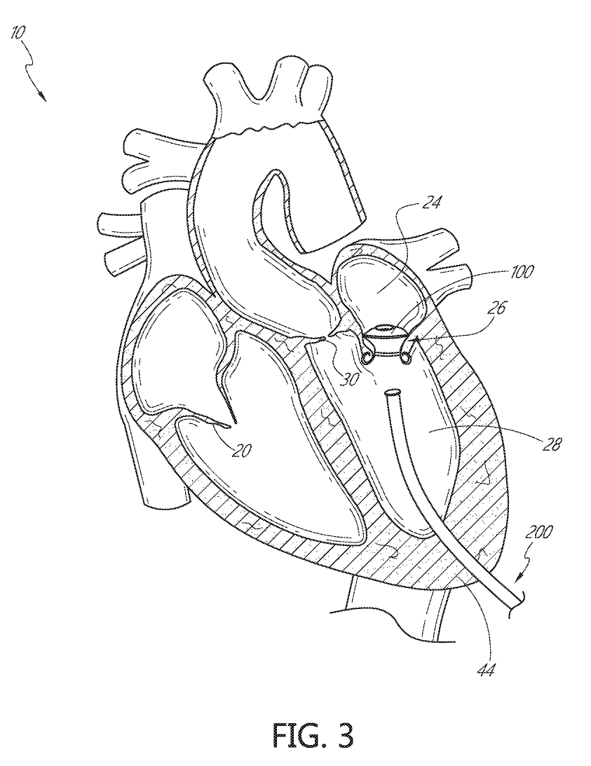

Referring now to FIG. 3, a heart 10 is shown in cross section to depict a placement of a cardiovascular prosthetic valve implant 100 in accordance with a non-limiting illustrative embodiment of the present disclosure. The illustrated implant 100 is shown spanning the native abnormal or diseased mitral valve. The implant 100 and various modified embodiments thereof will be described in detail below. As will be explained in more detail below, the implant 100 can be delivered to the heart trans-apically using a delivery catheter 200. In some variants, the delivery catheter 200 and implant 100 can be configured to deliver the implant 100 minimally invasively using an intravascular approach. In certain embodiments, the delivery catheter 200 and implant 100 can be configured to deliver the implant 100 transatrially through an incision in the wall of the left atrium 24. As illustrated in FIG. 3, the inflow portion of the implant 100 can sit in the left atrium 24, and the outflow portion of the implant can reside in the left ventricle 28. In the illustrated embodiment, the implant 100 can be placed over the native abnormal or diseased mitral valve 26. In other arrangements, the native abnormal or diseased mitral valve 26 can be partially or completely removed before implanting the valve 100.

### Implant

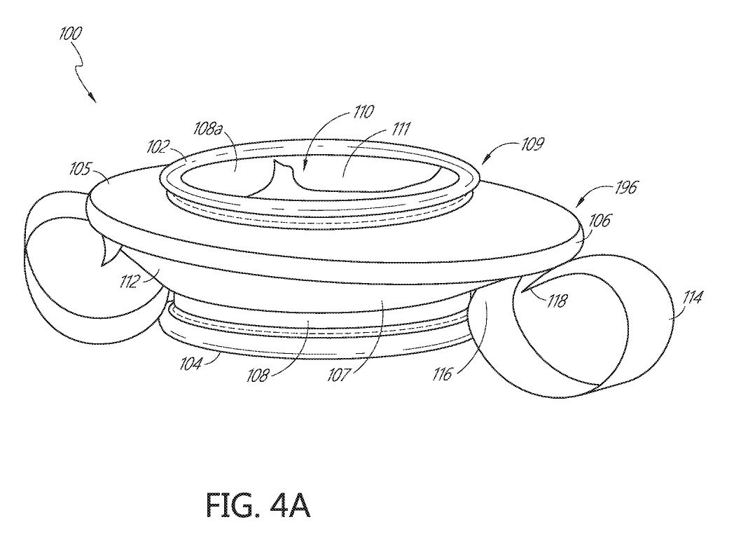

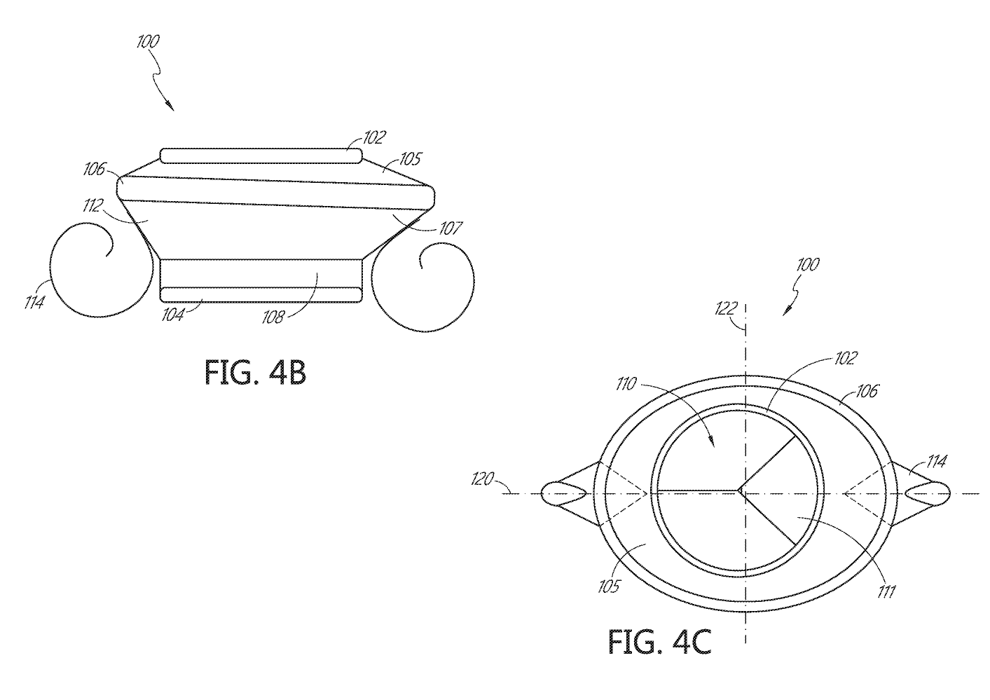

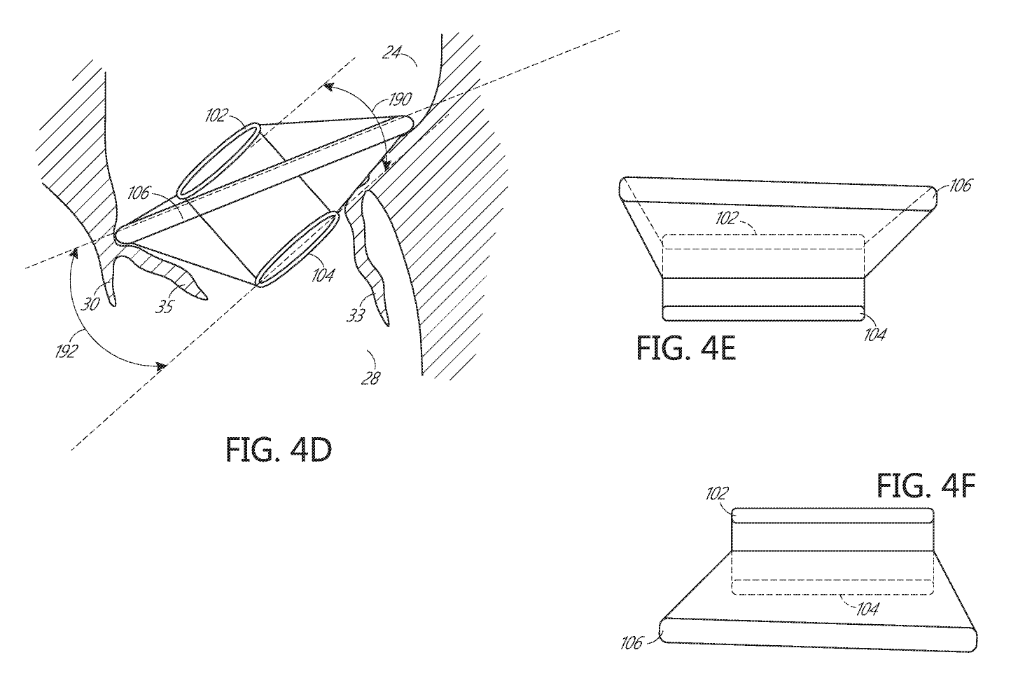

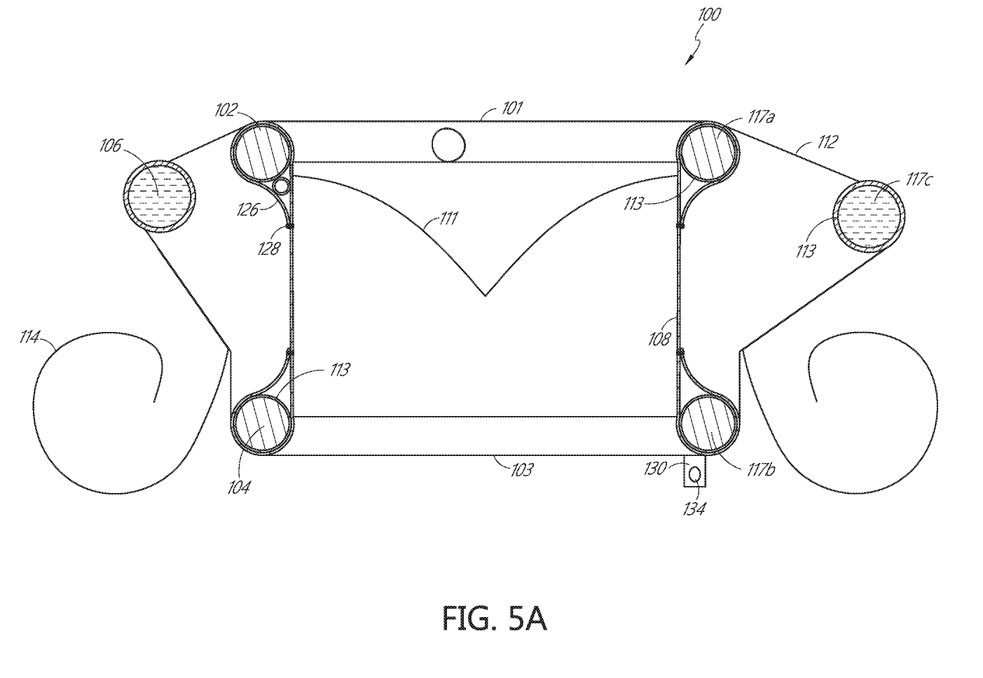

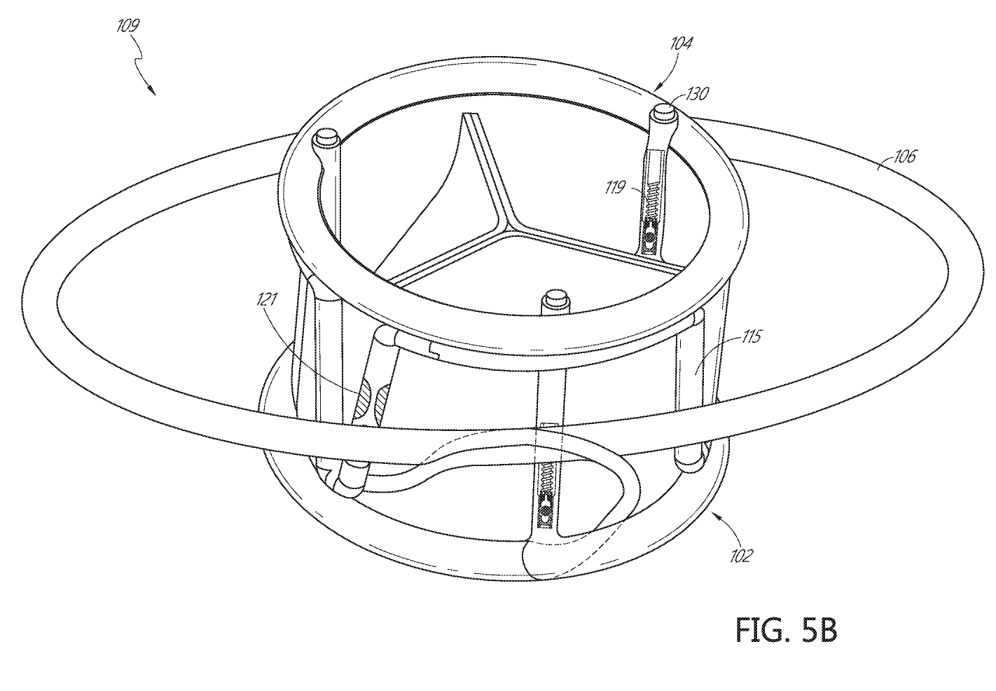

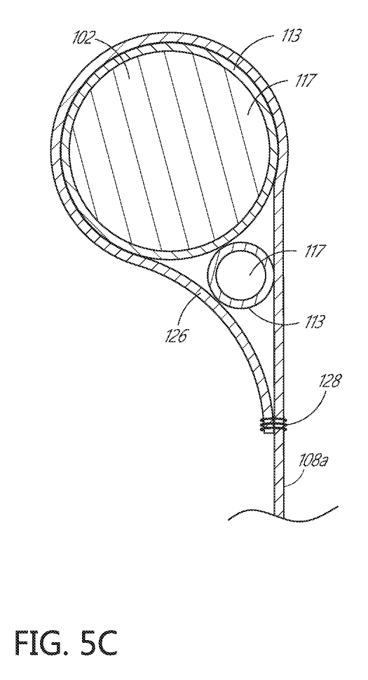

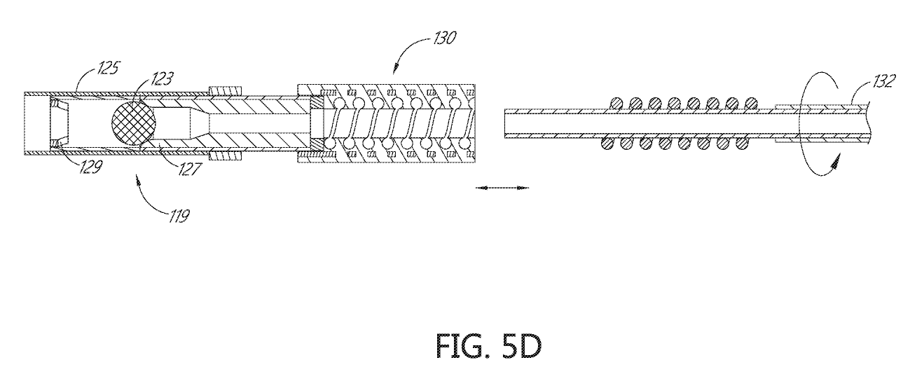

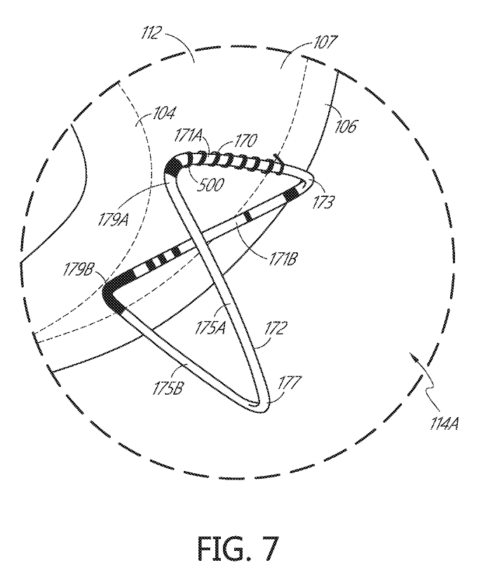

In some embodiments, the implant 100 can be a cardiovascular prosthetic valve implant and in some embodiments a prosthetic mitral valve implant. With reference to FIG. 4A, the implant 100 can have a shape that can be viewed as a generally tubular member with a flange portion that extends radially beyond an inner valve member. The implant 100 can include an inflatable structure 109 (shown in FIG. 5B), which in the illustrated embodiment includes an inflow ring 102, an outflow ring 104, and/or an atrial ring 106. While the implant 100 is often shown as having an inflow ring 102, an outflow ring 104, and an atrial ring 106, the implant 100 need not include all of these rings. In some embodiments, the implant 100 can have only one or only two of the aforementioned rings. In addition, some embodiments of the inflatable structure 109 can be formed without the illustrated struts 115 (described below). In addition, while main embodiments are described and show the implant 100 including anchors 114 (described below), some embodiments of the implant 100 may not include anchors 114. As shown in FIG. 5A, the inflow ring 102, outflow ring 104, and atrial ring 106 can be formed by tubular members 113 that can form channels 117 through which inflation media can be injected to inflate the inflatable structure 109. In this manner, certain embodiments, the inflow ring 102, outflow ring 104, and atrial ring 106 are inflatable as described herein. In certain arrangements, the implant 100 can be inflated with an inflation media that does not solidify (e.g., saline and/or air), thereby allowing the inflation media to be removed later from the implant 100. In such arrangements, the inflation media can provide a temporary structure to the implant 100 during which the function and/or positioning of the valve 100 can be evaluated, tested and/or adjusted. As discussed below, in certain arrangements, the implant 100 can be inflated with an inflation media that solidifies (e.g., epoxy), allowing the implant 100 to have a more rigid supporting structure after the inflation media solidifies. While in many of the embodiments described herein, components of the inflatable structure such as the inflow ring 102, outflow ring 104, and/or atrial ring 106 are inflatable and the valve 100 does not include a stent and can be stentless, in certain arrangements one or more of the inflow ring 102, outflow ring 104, and/or atrial ring 106 can be formed or include a non-inflatable support component such as a circumferential stent or ring that can be self-expanding and/or balloon expanded and in certain embodiments can be made of a metal. In some embodiments, the rings 102, 104, 106 can be inflated independent of one another, as described later. When the rings 102, 104, 106 are in a deflated state, the implant 100 can be compactly stored in the delivery catheter 200, which will be described in more detail below with reference to FIGS. 7A-7D. The rings 102, 104, 106 can be inflated when the implant 100 is deployed, thereby allowing the implant 100 to be seated in the valve annulus, as described below.

With continued reference to FIG. 4A, the implant 100 can include a cuff or body 108 that extends between the inflow and outflow rings 102, 104. The cuff 108 can be adapted to support a valve 110 that is coupled to the cuff 108. The cuff 108 can be tubular with an inflow end and an outflow end corresponding to the inflow ring 102, outflow ring 104. An inner surface 108a of the cuff 108 can define a flow path through which blood can flow through the implant 100. The valve 110 can include one or more leaflets 111 positioned in the flow path defined by the inner surface 108a of the cuff 108. In some embodiments, the valve 110 is a tissue valve comprising one or more leaflets 111 that can be stitched or otherwise coupled at their ends to the cuff 108. In some embodiments, the leaflets 111 of the tissue valve have a thickness equal to or greater than about 0.011 inches. In some embodiments, the tissue valve has a thickness equal to or greater than about 0.018 inches. As will be explained in more detail below, the valve 110 can be configured to move in response to the hemodynamic movement of the blood pumped by the heart 10 between an “open” configuration where blood can flow through the implant 100 in a first direction and a “closed” configuration whereby blood is prevented from back flowing through the valve 110 in a second direction. For example, the valve 110 of the illustrated implant 100 can allow blood flow in the direction from the inflow ring 102 to the outflow ring 104 but prevent flow in the direction from the outflow ring 104 to the inflow ring 102. In some embodiments, the implant 100 can include a valve structure that has already been proven to have acceptable clinical characteristics (e.g., flow performance, wear performance) in another valve of the heart and/or for the mitral valve and/or for which clinical data exists. For example, the implant 100 can include the inflow and outflow rings 102, 104, the cuff 108, and the leaflet subassembly of a prosthetic aortic valve that has already been cleared for use in humans and/or for which clinical data has already been collected on the aortic valve. Thus, certain embodiments can include testing clinical characteristics (e.g., flow performance, wear performance of the valve structure on one valve of the heart (e.g., aorta) and then using the same valve structure as part of an implant configured for another valve (e.g., mitral) of the heart. Additional embodiments of the valve 110 and the leaflet 111 subassembly, can be found in U.S. Patent Publication No. 2012/0016468 to Robin et al., the disclosures of which are expressly incorporated by reference in their entirety herein.

In the illustrated embodiment, the cuff 108 can comprise a thin flexible tubular material such as a flexible fabric or thin membrane with little dimensional integrity. As will be explained in more detail below, the cuff 108 can be changed preferably, in situ, to a support structure to which other components (e.g., the valve 110) of the implant 100 can be secured and where tissue ingrowth can occur. When the inflatable structure 109 of the valve 110 is uninflated, the cuff 108 is preferably incapable of providing support. The cuff 108 can be made from many different materials such as Dacron, TFE, PTFE, ePTFE, woven metal fabrics, braided structures, polyester fabric, or other generally accepted implantable materials as seen in conventional devices such as surgical stented or stentless valves and annuloplasty rings. These materials may also be cast, extruded, or seamed together using heat, direct or indirect, sintering techniques, laser energy sources, ultrasound techniques, molding or thermoforming technologies. The fabric thickness of the cuff 108 may range from about 0.002 inches to about 0.020 inches depending upon material selection and weave. Weave density may also be adjusted from a very tight weave to prevent blood from penetrating through the fabric to a looser weave to allow tissue to grow and surround the fabric completely. In certain embodiments, the fabric may have a thickness of at least about 20 denier.

As shown in FIG. 4A and FIG. 5A, the implant 100 can include a skirt 112 that extends from the atrial ring 106 to the cuff 108. The skirt 112 can have a top portion 105 that faces the inflow ring 102 and a bottom portion 107 that faces the outflow ring 104. The skirt 112 can be made from many different materials, weaves, and thicknesses, as discussed above for the cuff 108. In some variants, the bottom portion 107 of the skirt 112 can be adapted to exclude the native valve 26 or vessel. In certain embodiments, the bottom portion 107 can be adapted to seal blood flow from re-entering the left atrium 24. The material and/or weave of the skirt 112 can be selected to permit blood to enter the space between the skirt 112 and the cuff 108, thereby allowing a clot to form within the skirt 112. The clot can assist in seating and/or sealing the implant 100 within the valve annulus. In some embodiments, the skirt 112 can be adapted to promote tissue ingrowth into the space between the skirt 112 and the cuff 108.

The bottom portion 107 of the skirt 112 can exclude the native valve 26 or can extend over the former location of the native valve 26 and replace its function. The lower portion 107 can have an appropriate size and shape so that it does not interfere with the proper function of a neighboring valve (e.g., aortic valve 30) and/or does not impede blood flow through the left ventricular outflow tract (LVOT). In certain aspects, the lower portion 107 can be adapted so that the lower portion 107 does not interfere with clearance of blood behind the leaflets of the native mitral valve 26. If the lower portion 107 extends too far into the left ventricle 28, the implant 100 may restrain the mitral valve 26 near the wall of the left ventricle 28, creating a potential site for blood stagnation and thrombosis. By limiting the extension of the lower portion 107 into the left ventricle 28, the implant 100 can allow the apical portions of the mitral valve leaflets to move during the cardiac cycle, thereby flushing the blood out from this potential site of thrombosis.

As mentioned above, the implant 100 can include one or more features for grasping or attaching the implant 100 to the fibrotic tissue that surrounds the annulus of the mitral valve 26. Referring to FIG. 4A, the implant 100 can include one or more anchors 114. The anchors 114 can have a base portion 116 and a tip portion 118. The base portion 116 can be attached to the bottom portion 107 of the skirt 112. In some variants, the anchors 114 are diamond-shaped with one point of the diamond attached to the bottom portion 107 near the atrial ring 106. The anchors 114 can have an unconstrained configuration in which the tip portion 118 curls toward the base portion 116, as shown in FIG. 4A, to form a spiral configuration, which as explain below can be configured in certain arrangements to grasp or attach to the trigones 40, 42. The anchors 114 can be configured so that there is a negative clearance between the tip portion 118 and the atrial ring 106 when the anchor 114 is in its expanded configuration, which in the illustrated embodiment is a spiral configuration. In other words, the tip portion 118 can collide with the atrial ring 106 or a portion of the skirt 112 when the anchor 114 is unconstrained. In some embodiments, the anchor 114 and atrial ring 106 can function as a pressure-release valve such that if ventricular pressures become excessive the anchor 114 can unfurl slightly, thereby causing the atrial ring 106 to unseat from the native valve annulus and allowing blood to flow over the outer surface of the implant 100 and into the left atrium 24. This pressure-release function would not occur often but can allow the implant 100 to remain in place under conditions of excessive ventricular pressure. This pressure-release function can be preferable to having the implant 100 migrate into the left atrium 24.

The anchors 114 can be flexible and can be forced into a linear configuration that reduces the profile of the anchor 114 when the implant 100 is loaded into the delivery catheter 200, as described below. In some variants, the anchors 114 can be adapted to capture at least a portion of the left and right trigones 40, 42 (shown in FIG. 2), as discussed below. The anchors 114 can be positioned on the implant 100 so that anchors 114 allow normal movement of the native leaflets. For example, the anchors 114 can be substantially aligned with the plane of coaptation for the posterior and anterior leaflets 33, 35 of the mitral valve 26, thereby allowing the anchors 114 to attach to the trigones 40, 42 while avoiding having the anchors 114 interfere with normal movement of the posterior and anterior leaflets 33, 35. The anchors 114 can include a wire form structure that is embedded in a material such as the material that is used to make the cuff 108 or skirt 112. The anchors 114 can be coupled to the lower portion 107 of the valve 107 in several manners such as by adhesive, staples, stitching etc.

In certain arrangements, the axial stabilization of the implant 100 can be established by the combined effects of the atrial ring 106 and the anchors 114. The atrial ring 106 can be designed to sit on the atrial aspect of the mitral valve annulus and can be preferably shaped in such a way that it maintains good apposition with the mitral valve annulus. The atrial ring 106 can be sized to prevent the implant 100 from migrating into the left ventricle 28 and to prevent blood from back flowing around the outer surface of the implant 100. In certain arrangements, the atrial ring 106 of the implant 100 can be flexible and can conform to the native anatomical atrial ring when inflated. For example, in one arrangement, atrial ring 106 of the implant 100 is initially flexible when inflated with a non-solidifying inflation media (e.g., saline, gas) or with a solidifying inflation media (e.g., epoxy) that has not yet solidified. Thus, the atrial ring 106 can conform to the native anatomical atrial ring initially and retain that conformity after the non-solidifying media is displaced with a solidifying inflation media or after the solidifying inflation media solidifies. In this way, the implant 100 can allow the valve 100 to be formed in place or in situ to conform to the anatomy. In addition, in certain embodiments, the skirt 112 can be flexible and aid the implant in conforming to the native anatomical atrial ring. The atrial ring 106 and skirt 112 can from an atrial flange 196. The atrial flange 196 can have a smooth surface with blunt edges. The atrial flange 196 can be designed so that the atrial flange 196 does not have sharp edges that could abrade, cut, dig into, or otherwise damage surrounding heart tissue that contacts the atrial flange 196. In some embodiments, all edges and/or exposed surfaces of the atrial flange 196 have a minimum radius of curvature of about 0.010″ in one arrangement, of about 0.030″ in one arrangement, and of about 0.100″ in one arrangement. In certain embodiments, the atrial flange 196 can have a height defined as the distance from the upper surface of the inflow ring 102 to the point of contact between the atrial flange 196 and the surrounding heart tissue. In some embodiments, the height of the atrial flange 196 is greater than about 3 mm, greater than about 5 mm, and greater than about 15 mm. In some embodiments, the atrial flange 196 can have a maximum height of about 20 mm. In some embodiments, the atrial flange 196 can have a height between about 3 mm and about 20 mm and in some embodiments between about 5 mm and about 20 mm and in some embodiments between about 15 mm and about 20 mm.

In certain embodiments, the solidifying inflation media can be a polymer that is designed such that as a liquid, the polymer has low viscosity for catheter delivery, cures at 37° C. with minimal change in temperature, allows fluoroscopic imaging during delivery, is soluble in blood in the liquid form, and does not form emboli. The polymer, once cured, can provide a structure with good mechanical and chemical stability in an aqueous environment and is biocompatible. In certain embodiments, the polymer can comprise five components in which two epoxides form an epoxy resin, two amines that combined act as a hardener and a fifth component that is a radiopaque compound to facilitate placement of the device.

The anchors 114 can be designed to capture the fibrotic tissue surrounding the mitral valve annulus from the ventricular aspect, thereby preventing the implant 100 from migrating into the left atrium 24. The curvature and elasticity of the anchors 114 can be adapted so that when the anchors 114 are deployed, the tip portion 118 of the anchors 114 grab surrounding tissue and pull the base portion 116 of the anchor 114 toward the tip portion 118, thereby pulling the atrial ring 106 against the annulus of mitral valve 26 and improving the seal between the implant 100 and the mitral valve annulus. In some embodiments, the cuff 108 can have a short longitudinal height because the sealing function of the implant 100 is performed by the atrial ring 106, the skirt 112, and the anchors 114. This can allow the valve 110 to have a short height. The valve 110 can have a height defined as the distance between the top surface of the inflow ring 102 and the bottom surface of the outflow ring 104. In some embodiments, the valve 110 can have a height between about 18 mm and about 20 mm. In certain variants, the valve 110 can have a height between about 8 mm and about 30 mm. A short valve height can minimize ventricular stasis, minimize obstruction of the LVOT, and allow treatment of a large range of patient anatomies. In some embodiments, the valve 110 can be biased toward the left atrium 24 to reduce the outflow ring 104 from obstructing native valve movement and/or blood flow through the LVOT, as discussed below. In certain embodiments, the portion of the implant 100 that resides in the left atrium 24 contains no metal and/or in certain embodiments no circumferential stent structures. In some embodiments, the outflow ring 104 can extend into the left ventricle by a longitudinal distance of no more than about 15 mm, of no more than about 10 mm, and of no more than about 5 mm. In some embodiments, the implant 100 can be configured so that no part of the valve 110 extends below the annulus of the native mitral valve 26, as shown in an illustrated embodiment of FIG. 4F.

In some variants, the shape of the implant 100 is preferably contoured to engage a feature of the native anatomy in such a way as to prevent the migration of the implant 100 in a proximal or distal direction. In one embodiment the feature that the implant 100 engages is the mitral valve annulus and/or the fibrotic tissue surrounding the valve annulus. In certain embodiments, the feature that the implant 100 engages to prevent migration has a diameter difference between 1% and 10% with respect to the atrial ring 106. In another embodiment, the feature that the implant 100 engages to prevent migration has a diameter difference between 5% and 40% with respect to the atrial ring 106. In certain embodiments the diameter difference is defined by the free shape of the implant 100. In another embodiment the diameter difference prevents migration in only one direction. In another embodiment, the diameter difference prevents migration in two directions, for example the retrograde and antegrade directions. In certain embodiments, the atrial flange of the implant 100 can vary in diameter ranging from about 40 mm by 50 mm to about 60 mm by 100 mm the inside diameter of the portion of the structure that holds the valve will be between about 20 mm and about 26 mm and can have a height ranging from about 16 mm to about 22 mm in the portion of the implant 100 where the leaflets of the valve 110 are mounted. In some embodiments, the inside diameter of the portion of the structure that holds the valve can be between about 10 mm to about 45 mm. In some embodiments, the implant 100 can have an outside diameter of between about 30 mm and about 70 mm, or preferably between about 35 mm and about 60 mm when fully inflated. With reference to FIG. 4A, in the illustrated embodiment, each of the inflow ring 102 and the outflow ring 104 can have a cross-sectional diameter of about 0.090 inches. In some embodiments, the cross-sectional diameter of each of the inflow ring 102 and the outflow ring 104 can be between about 0.060 inches and about 0.120 inches.

Since the implant 100 can be inflated and may be placed without the aid of a dilatation balloon for radial expansion, the mitral valve 26 may, in certain arrangements, not have any duration of obstruction and can provide the patient more comfort and the physician more time to properly place the implant 100 accurately. Because the implant 100 is not utilizing a support member with a single placement option as a plastically deformable or shaped memory metal stent does, the implant 100 may be movable and or removable if desired. This could be performed multiple times until the implant 100 is permanently disconnected from the delivery catheter 200 as will be explained in more detail below. In addition, the implant 100 can include features, which allow the implant 100 to be tested for proper function, sealing and sizing, before the delivery catheter 200 is disconnected. In addition, because the annulus of the mitral valve 26 changes shape and orientation throughout the cardiac cycle, the atrial ring 106 of the implant 100 can be better suited to track and seal with the annulus compared with a plastically deformable or shaped memory metal stent. The inflatable implant 100 can also better resist fatigue from repetitive elastic loading compared with a shaped memory metal stent. In certain embodiments, the skirt 112 can conform (at least partially) to the anatomy of the patient as the implant 100 is inflated. Such an arrangement may provide a better seal between the patient's anatomy and the implant 100.

Referring to FIG. 4B, various shapes of the implant 100 can be manufactured to best fit anatomical variations from person to person. For example, the size and orientation of the atrial ring 106 can be selected to match the geometry of the mitral valve annulus of the patient. The shape of the implant 100 can include a simple cylinder, a hyperboloid, a device with a larger diameter in its mid portion and a smaller diameter at one or both ends, a funnel type configuration or other conforming shape to native anatomies. In the illustrated embodiment, the atrial ring 106 is in a plane declined about 7° with respect to a plane containing the inflow ring 102. The angle of the atrial ring 106 relative to the inflow ring 102 can be selected so that when the atrial ring 106 is seated on annulus of the mitral valve 26, the angle of the implant valve 110 relative to the aortic-mitral curtain 36 matches the anatomy of the native valve 26. In certain embodiments, the atrial ring 106 can be in a plane that can decline with respect to a plane containing the inflow ring 102 by an angle ranging between about 1° and about 15°. In the illustrated embodiment, the inflow ring 102 is substantially parallel to the outflow ring 104. In some variants, the inflow ring 102 can be at an angle with respect to the outflow ring 104. In certain embodiments, the top and bottom portions 105, 107 of the skirt 112 are configured to determine the orientation of the atrial ring 106 when the atrial ring 106 is inflated.

Referring to FIG. 4C, the atrial ring 106 can be ellipse-shaped and slightly off-center relative to the inflow and outflow rings 102, 104. The inflow and outflow rings 102, 104 can be axially aligned with one another. In the illustrated embodiment, the atrial ring 106 is an ellipse shape with major and minor diameters of 50 mm and 40 mm. However, the implant 100 can have other configurations. For example, the atrial ring 106 can be circular or polygonal. In addition, the inflow and outflow rings 102, 104 need not be axially aligned with one another. In some embodiments, the inflow and/or outflow rings 102, 104 can be ellipse-shaped or polygonal. Although the illustrated embodiment shows the atrial ring 106 tilted with respect to the minor axis 122, the atrial ring 106 can be tilted with respect to the major axis 120 or any other axis. In other words, the point on the atrial ring 106 that is closest to the inflow ring 102 can be anywhere on the perimeter of the atrial ring 106.

In the illustrated embodiment, as noted above, the implant 100 includes a pair of anchors 114 that can be spaced circumferentially 180° apart from one another and are aligned along a major axis 120 of the atrial ring 106. However, the anchors 114 can take other configurations. For example, the implant 100 can include none, one, two, or more than two anchors 114. The anchors 114 can be unevenly distributed circumferentially around the atrial ring 106. The anchors 114 can be aligned along a minor axis 122 of the atrial ring 106. The anchors 114 can be positioned on the implant 100 at a location other than the major or minor axis 120, 122 of the atrial ring 106. The anchors 114 can be designed to atraumatically capture tissue. For example, the tip of the anchors can be blunt. In some embodiments, the anchors 114 can pierce tissue. For example, the anchors may include hooks or pointed features.

Referring to FIG. 4D, the implant 100 can be tailored so that the inflow ring 102 is at an angle relative to the atrial ring 106, as mentioned above. The inflow ring 102 can form and inflow angle 190 relative to the atrial ring 106. In some variants, the inflow angle 190 can be between about 0° and about 60°. In addition, the implant 100 can be designed so that the outflow ring 104 is at an angle relative to the inflow ring 102 and/or the atrial ring 106. The outflow ring 104 can form and outflow angle 192 relative to the atrial ring 106. In some variants, the outflow angle 192 can be between about 0° and about 60°. In some embodiments, the outflow angle 192 can be selected so that the outflow ring 104 contacts the posterior leaflet 33 of the mitral valve 26 near the base of the leaflet, thereby allowing the apical portion of the posterior leaflet 33 to move during the cardiac cycle and flush blood from the site of potential flow stagnation near the ventricular wall. In addition, as mentioned above, the outflow ring 104 can be biased toward the left atrium 24 to minimize the outflow ring 104 obstructing movement of the posterior valve 33 and/or blood flow through the LVOT. In addition, the implant 100 can be made with the relative angles between that the inflow ring 102, the atrial ring 106 and/or the outflow ring 104 described above and/or because of the flexible structure of the cuff 108, the skirt 112 and/or the inflatable structure 109, the tailoring of the relative angles between that the inflow ring 102, the atrial ring 106 and/or the outflow ring 104 described can be done in-situ as the inflatable structure 109 is hardened and fixes the relationships between the inflow ring 102, the atrial ring 106 and/or the outflow ring 104.

In some embodiments, the inflow ring 102 can be longitudinally interposed between the atrial ring 106 and the outflow ring 104, as shown in FIG. 4E. In some embodiments, the atrial ring 106 and the inflow ring 102 longitudinally overlap with one another. In other words, the implant 100 can be configured so that the inflow ring 102 is partially or completely downstream of the atrial ring 106. In certain variants, the outflow ring 104 can be partially or completely upstream of the atrial ring 106, as shown in FIG. 4F. In addition, one more of the rings 102, 104, 106 can be saddle-shaped. For example, in some variants, the atrial ring 106 can be curved so that the portion of the atrial ring 106 that faces the left atrium 24 is concave.

With reference to FIGS. 5A-C, in the illustrated embodiment, the implant 100 can include the inflatable structure 109 that is formed by the one or more inflatable rings 102, 104, 106, and, in the illustrated embodiment, one or more struts 115. The inflatable rings 102, 104, 106 can be formed by a number of distinct tubular members 113 (e.g., balloon rings or toroids). In the illustrated embodiment, the implant 100 comprises an inflow ring 102 at a top surface 101 of the cuff 108, an outflow ring 104 at a bottom surface 103 of the cuff 108, and an atrial ring 106 disposed intermediate of the inflow and outflow rings 102, 104. The inflow and outflow rings 102, 104 can be secured to the cuff 108 in any of a variety of manners. With reference to FIGS. 5A and 5C, in the illustrated embodiment, the inflow and outflow rings 102, 104 can be secured within folds 126 formed at the top surface 101 and the bottom surface 103 of the cuff 108. The folds 126, in turn, are secured by sutures or stitches 128. When inflated, the implant 100 is supported in part by the inflow and outflow rings 102, 104 pulling the cuff 108 taut and the atrial ring 106 pulling the skirt 112 taut. The rings 102, 104, 106 and struts 115 can form one or more inflatable channels 117 that can be inflated by air, liquid or inflation media. The rings 102, 104, 106 can include distinct inflatable channels 117a, 117b, 117c, thereby allowing each inflatable channel 117 or ring 102, 104, 106 to be inflated independently of the other rings 102, 104, 106. As noted above, while in many of the embodiments described herein, components of the inflatable structure such struts 115 are inflatable, in certain arrangements one or more of the struts 115, inflow ring 102, outflow ring 104, and/or atrial ring 106 can be formed or include a non-inflatable support component such as a wire, bar or circumferential stent that can be self-expanding and/or balloon expanded and can be made of a metal

The inflation media that is inserted into the inflation channels 117 and/or ring 102, 104, 106 can be pressurized and/or can solidify in situ to provide structure to the implant 100. The inflatable structure 109 can be inflated using any of a variety of inflation media, depending upon the desired performance. In certain embodiments, the inflation media can include a liquid such water or an aqueous based solution, a gas such as CO₂, or a hardenable media which may be introduced into the inflation channels 117 at a first, relatively low viscosity and converted to a second, relatively high viscosity. Viscosity enhancement may be accomplished through any of a variety of known UV initiated or catalyst initiated polymerization reactions, or other chemical systems known in the art. The end point of the viscosity enhancing process may result in a hardness anywhere from a gel to a rigid structure, depending upon the desired performance and durability. In certain arrangements, useful inflation media generally include those formed by the mixing of multiple components and that have a cure time ranging from a tens of minutes to about one hour, an in certain embodiments, from about twenty minutes to about one hour. Such a material may be biocompatible, exhibit long-term stability (for example, on the order of at least ten years in vivo), pose as little an embolic risk as possible, and exhibit adequate mechanical properties, both pre and post-cure, suitable for service in the cuff in vivo. For instance, such a material could have a relatively low viscosity before solidification or curing to facilitate the cuff and channel fill process. In certain embodiments, a desirable post-cure elastic modulus of such an inflation medium is from about 50 to about 400 psi-balancing the need for the filled body to form an adequate seal in vivo while maintaining clinically relevant kink resistance of the cuff. The inflation media can be radiopaque, both acute and chronic. Other embodiments of the inflation media can be found in U.S. Patent Publication No. 2012/0022629 to Perera et al., the disclosures of which are expressly incorporated by reference in their entirety herein.

Since the inflation channels 117 generally surround the cuff 108, and the inflation channels 117 can be formed by separate tubular members 113 (e.g., balloons), the attachment or encapsulation of these inflation channels 117 can be in intimate contact with the cuff material. In some embodiments, the inflation channels 117 are encapsulated in the folds 126 or lumens made from the cuff material sewn to the cuff 108, as shown in FIG. 5C. These inflation channels 117 can also be formed by sealing the cuff material to create an integral lumen from the cuff 108 itself. For example, by adding a material such as a silicone layer to a porous material such as Dacron, the fabric can resist fluid penetration or hold pressures if sealed. Materials may also be added to the sheet or cylinder material to create a fluid-tight barrier.

In some embodiments, the implant 100 is not provided with separate tubular members 113, instead the fabric of the cuff 108 and/or the skirt 112 can form the inflation channels 117. For example, in one embodiment two fabric tubes of a diameter similar to the desired final diameter of the implant 100 are placed coaxial to each other. The two fabric tubes are stitched, fused, glued or otherwise coupled together in a pattern of channels 117 that is suitable for creating the geometry of the inflatable structure 109. In some embodiments, the fabric tubes are sewn together in a pattern so that the ends of the fabric tubes form an annular ring or toroid (e.g., inflow ring 102). In some embodiments, the middle section of the implant 100 contains one or more inflation channels 117 shaped in a step-function pattern. In some embodiments, the fabric tubes are sewn together at the middle section of the implant 100 to form inflation channels 117 that are perpendicular to the end sections of the implant 100. Additional embodiments of methods for fabricating certain components of the implant 100 can be found in U.S. Patent Publication No. 2006/0088836 to Bishop et al., the disclosure of which are expressly incorporated by reference in their entirety herein.

With particular reference to FIG. 5B, in the illustrated embodiment, the inflow ring 102 and struts 115 can be joined such that the inflation channel 117 of the inflow ring 102 is in fluid communication with the inflation channel 117 of some of the struts 115. The inflation channel 117 of the outflow ring 104 can also be joined in communication with the inflation channels 117 of the outflow ring 104 and a few of the struts 115. In the illustrated embodiment, the atrial ring 106 is in fluid communication with the inflation channel 117 of the inflow ring 102. In some variants, the atrial ring 106 is in communication with some of the struts 115 but is isolated from the inflow and outflow rings 102, 104. In this manner, the inflation channels 117 of the (i) inflow ring 102 and a few struts 115 can be inflated independently from the (ii) outflow ring 104 and some struts 115. In some embodiments, the inflation channel 117 of the inflow ring 102 is in communication with the inflation channel of the struts 115, while the inflation channel 117 of the outflow ring 104 is not in communication with the inflation channel 117 of the struts 115. The skilled artisan will appreciate that the inflation channels 117 can be arranged to allow the rings and/or struts to be inflated in series with or independent of other components of the inflatable structure 109. As will be explained in more detail below, the two groups of inflation channels 117 can be connected to independent PFL tubing 132 to facilitate the independent inflation of the channels 117. It should be appreciated that in modified embodiments the inflatable structure 109 can include less (i.e., one common inflation channel 117) or more independent inflation channels 117. For example, in one embodiment, the inflation channels 117 of the inflow ring 102, struts 115 and outflow ring 104 can all be in fluid communication with each other such that they can be inflated from a single inflation device. In another embodiment, the inflation channels 117 of the inflow ring 102, struts 115 and outflow ring 104 can all be separated and therefore utilize three inflation devices.

With reference to FIG. 5B, in the illustrated embodiment, each of the inflow and outflow rings 102, 104 can have in certain arrangements a cross-sectional diameter of about 0.090 inches. The struts 115 can have a cross-sectional diameter of about 0.060 inches. In some embodiments, within the inflation channels 117 are also housed valve systems that allow for pressurization without leakage or passage of fluid in a single direction. In the illustrated embodiment shown in FIG. 5B, two end valves or inflation valves 119 reside at each end section of the inflation channels 117 adjacent to the connection ports 130. The connection ports 130 can be positioned radially inward of the outer diameter of the inflow and/or outflow rings 102, 104, as shown in FIG. 5B. In some variants, the connection ports 130 can be positioned radially outward of the outer diameter of the inflow and/or outflow rings 102, 104. In some embodiments, the PFL tubes 132 access the connection ports 130 through holes in the portion of the skirt 112 that covers the bottom surface 103 of the implant 100. The end valves 119 can be utilized to fill and exchange fluids such as saline, contrast agent and inflation media. The length of this inflation channel 117 may vary depending upon the size of the implant 100 and the complexity of the geometry. The inflation channel material may be blown using heat and pressure from materials such as nylon, polyethylene, Pebax, polypropylene or other common materials that will maintain pressurization. The fluids that are introduced are used to create the support structure 109, where without the fluids, the implant 100 is an undefined fabric and tissue assembly. In one embodiment the inflation channels 117 are first filled with saline and contrast agent for radiopaque visualization under fluoroscopy. This can make positioning the implant 100 at the implantation site easier. This fluid is introduced from the proximal end of the catheter 200 with the aid of an inflation device such as an endoflator or other means to pressurize fluid in a controlled manner. This fluid is transferred from the proximal end of the catheter 200 through the PFL tubes 132 which are connected to the implant 100 at the end of each inflation channel 117 at the connection port 130.

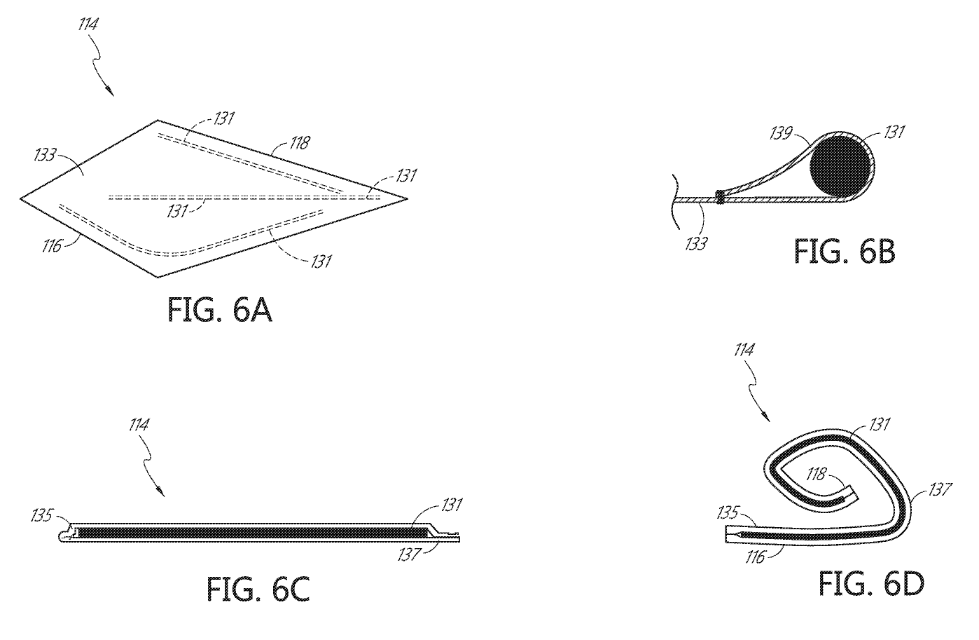

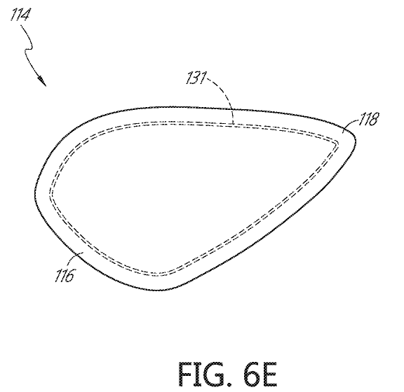

With continued reference to FIG. 5A, the inflation channels 117 can be configured so that the cross-sectional profile of the implant 100 is reduced when it is compressed or in the retracted state. The inflation channels 117 can be arranged in a step-function pattern. The inflation channels 117 can have three connection ports 130 for coupling to the delivery catheter 200 via position and fill lumen tubing (PFL) tubing 132 (see FIGS. 6A and 6C). In some embodiments, at least two of the connection ports 130 also function as inflation ports, and inflation media, air or liquid can be introduced into the inflation channel 117 through these ports. The PFL tubing 132 can be connected to the connection ports 117 via suitable connection mechanisms. In one embodiment, the connection between the PFL tubing 132 and the connection port 117 is a screw connection. In some embodiments, an inflation valve 134 can be present in the connection port 130 and can stop the inflation media, air or liquid from escaping the inflation channels 117 after the PFL tubing 132 is disconnected. Additional details and embodiments of the connection ports 130, can be found in U.S. Patent Publication No. 2012/0016468 to Robin et al., the disclosures of which are expressly incorporated by reference in their entirety herein.

With reference to FIG. 5B, in the illustrated embodiment, the inflation channel 117 can have an end valve 119 (i.e., inflation valve) at each end whereby the inflation channel 117 can be separated from the PFL tubes 132 (shown in FIGS. 6A-6D) thus disconnecting the catheter 200 from the implant 100. This connection can be a screw or threaded connection, a colleting system, an interference fit or other devices and methods of reliable securement between the two components (i.e., the end valve 119 and the PFL tubes 132). In between the ends of the inflation channel 117 can be an additional directional valve 121 to allow fluid to pass in a single direction. This allows for the filling of each end of the inflation channel 117 and displacement of fluid in a single direction. The implant 100 can include two connection ports 130, each having an end valve 119. A PFL tube 132 can be connected to each of the two connection ports 130, thereby defining a flow loop in which media injected through the first PFL tube 132 can flow through the inflatable structure 109 and exit the inflatable structure 109 through the second PFL tube 132. By placing a directional valve 121 within the flow loop connecting the two connection ports 130, the components of the inflatable structure 109 can be selectively inflated. For example, the directional valve 121 can allow flow in the direction of a first connection port 130 to a second connection port 130 but not in the direction of the second connection port 130 to the first connection port 130. By injecting fluid inflation media at the first connection port 130, the components of the inflatable structure 109 can be inflated in series because the directional valve 121 allows flow to pass to the downstream components of the inflatable structure 109. By injecting media at the second connection port 130, only the components of the inflatable structure 109 that are downstream of the directional valve 121 will be inflated because the directional valve 121 blocks the media from reaching the upstream components. In this way, the implant 100 can include connection ports 130 and directional valves 121 that allow select portions of the inflatable structure 109 to be inflated independently of other portions of the inflatable structure 109. In some embodiments, the atrial ring 106 can be inflated independently of the inflow and outflow rings 102, 104. In some variants, the atrial ring 106 is inflated in series with the inflow ring 102 while the outflow ring 104 and/or struts 115 are inflated independently of the inflow ring 102 and atrial ring 106.

Once the implant 100 is placed at the desired position and inflated with saline and contrast agent, this fluid can be displaced by an inflation media that can solidify or harden. As the inflation media can be introduced from the proximal end of the catheter 200, the fluid containing saline and contrast agent is pushed out from one end of the inflation channel 117. Once the inflation media completely displaces the first fluid, the PFL tubes 132 can then be disconnected from the implant 100 while the implant 100 remains inflated and pressurized. The pressure can be maintained in the implant 100 by the integral valve (i.e., end valve 119) at each end of the inflation channel 117. In the illustrated embodiment depicted in FIG. 5D, the end valve 119 can have a ball 123 and seat to allow for fluid to pass when connected and seal when disconnected. In some cases the implant 100 has three or more connection ports 130, but only two have inflation valves 119 attached. The connection port 130 without the end valve 119 can use the same attachment device such as a screw or threaded element. Since, in the illustrated embodiment, this connection port 130 is not used for communication with the support structure 109 and its filling, no inflation valve 119 is necessary. In other embodiments, all three connection ports 130 can have inflation valves 119 for introducing fluids or inflation media.

With reference to FIG. 5D, the end valve system 119 can comprise a tubular section 125 with a soft seal 127 and spherical ball 123 to create a sealing mechanism. The tubular section 125 in one embodiment is about 0.5 cm to about 2 cm in length and has an outer diameter of about 0.010 inches to about 0.090 inches with a wall thickness of about 0.005 inches to about 0.040 inches. The material can include a host of polymers such as nylon, polyethylene, Pebax, polypropylene or other common materials such as stainless steel, Nitinol or other metallic materials used in medical devices. The soft seal material can be introduced as a liquid silicone or other material where a curing occurs thus allowing for a through hole to be constructed by coring or blanking a central lumen through the seal material. The soft seal 127 can be adhered to the inner diameter of the wall of the tubular member 125 with a through hole for fluid flow. The spherical ball 123 can move within the inner diameter of the tubular member 125 where it seats at one end sealing pressure within the inflation channels 117 and is moved the other direction with the introduction of the PFL tube 132 but not allowed to migrate too far as a stop ring or ball stopper 129 retains the spherical ball 123 from moving into the inflation channel 117. As the PFL tube 132 is screwed into the connection port 130, the spherical ball 123 is moved into an open position to allow for fluid communication between the inflation channel 117 and the PFL tube 132. When disconnected, the ball 123 can move against the soft seal 127 and halt any fluid communication external to the inflation channel 117 leaving the implant 100 pressurized. Additional embodiments can utilize a spring mechanism to return the ball to a sealed position and other shapes of sealing devices may be used rather than a spherical ball. A duck-bill style sealing mechanism or flap valve can also be used to halt fluid leakage and provide a closed system to the implant. Additional embodiments of end valve systems have been described in U.S. Patent Publication No. 2009/0088836 to Bishop et al., which is thereby incorporated by reference herein.

Referring to FIGS. 6A-6D, the anchors 114 can include one or more core members 131 that drive the anchors 114 into a spiral or deployed configuration. The core member 131 can be a flexible piece of plastic or metal (e.g., shape memory alloy such as nitinol) that is enclosed within a casing 133 of the anchor 114. In some embodiments, the casing 133 can include a top sheet 135 that is sewn or otherwise attached to a bottom sheet 137, with the core 131 being sandwiched between the top and bottom sheets 135, 137. In some embodiments, the core 131 can be encased within a fold 139 that is formed by wrapping the casing 133 around the core 131 and stitching or otherwise attaching the casing 133 to itself, as shown in FIG. 6B. As discussed below, the core member 131 can be forced into a straight configuration (as shown in FIG. 6C) that reduces the profile of the anchor 114, allowing the implant 100 to be compactly stowed in a delivery catheter 200.

Referring to FIG. 6A, the anchor 114 can have a base portion 116 for attaching the anchor 114 to the implant 100. The base portion 116 can be sewn, glued, welded, or otherwise attached to the implant 100. The base portion 116 can be attached to the lower portion 107 of the skirt, the inner surface of the cuff 108a, and/or the outer surface of the cuff 108. The anchor 114 can have a tip portion 118. The tip portion 118 can be free to curl toward the base portion 116 in a spiral configuration when the anchor 114 is unconstrained, thereby allowing the tip portion 143 to wrap around or otherwise capture tissue, as shown in FIG. 6D. In some embodiments, the anchor 114 includes a core 131 that forms a loop that is substantially parallel with the perimeter of the anchor 114, as shown in FIG. 6E. In one embodiment, the core 131 can be formed from a piece of wire formed of a plastic or metal (e.g., shape memory alloy such as nitinol) and in the embodiment of FIG. 6E the wire can form a loop. The tip 118 of the anchor 114 can be blunt. The tip of the looped core 131 can roll up toward the base portion 116 when the anchor 114 is unconstrained.

With continued reference to FIG. 6A, the core 131 can have a variety of configurations. For example, the core 131 can be centrally located along a mid-line of the anchor. In some variants, the core 131 can be positioned long a lateral edge of the anchor 114. The core can extend from the base portion 141 into the tip portion. In some variants, the anchor 114 can include a plurality of cores 131. The cores 131 can be substantially parallel to one another or can be positioned in different orientations. The cores 131 can extend along a straight line or can be curved. In the illustrated embodiment, the anchor 114 is diamond shaped. However, the anchor 114 can be configured to have other shapes. For example, the tip portion 143 can have a plurality of fingers that each contain a core 131. The tip portion 143 can fan out or be blunted to atraumatically capture tissue. The anchor 114 can include an inflation channel that allows the core 131 to be changed between a straight and a spiral configuration.

FIG. 7 illustrates another embodiment of an anchor 114A that can be used with the implant 100 embodiments described herein. The anchor 114A can be similar to the anchor 114 described above except as described differently below. The features of the anchor 114A can be combined or included with the anchor 114 or any other embodiment discussed herein. As shown in FIG. 7, in the illustrated embodiment, the anchor 114A can comprise a base 170 and a leg 172. The base 170 can be sewn or otherwise attached to the bottom portion 107 of the skirt 112 of the implant 100. The leg 172 can be formed of a flexible material such as, for example, nitinol or a polymeric material, and can have a collapsed state in which the leg 172 is folded toward the base 170. In one arrangement, the anchor 114A can be formed from a single piece of wire that has its ends crimped together. In certain arrangements, the wire can be heat set into the configuration shown in FIG. 7. The leg 172 can be secured to the implant the collapsed state by a suture 500 or other securing device. Upon release from the securing device, the leg 172 can spring away from the base 170, thereby creating a support structure that helps seat the implant 100 in the native mitral valve and resists the implant 100 from migrating into the left atrium.

With continued reference to FIG. 7, the base 170 can have a first leg 171A and a second leg 171B. The first and second legs 171A, 171B can be joined to one another by a bridge 173. As shown in FIG. 7, the bridge 173 can include a bend that has an apex directed toward the atrial ring 106. In some configurations the first and second legs 171A, 171B are not joined together by a bridge 173. In some embodiments, the bridge 173 can have an apex that is directed toward the outflow ring 104. As shown in FIG. 7, the anchor 114A can include a first projecting member 175A and a second projecting member 175B. The first and second projecting members 175A, 175B can be joined to one another by a strut 177. As shown in FIG. 7, the strut 173 can include a bend having an apex that is directed away from the skirt 112. In some configurations the first and second projecting members 175A, 175B are not joined together by a strut 177. In some embodiments, the strut 177 can have an apex that is directed toward the skirt 112. In the illustrated embodiment, the first leg 171A is joined to the first projecting member 175A by a first joint 179A, and the second leg 171B is joined to the second projecting member 175B by a second joint 179B. In some configurations, the anchor 114A may have only one of the first and second joints 179A, 179B.

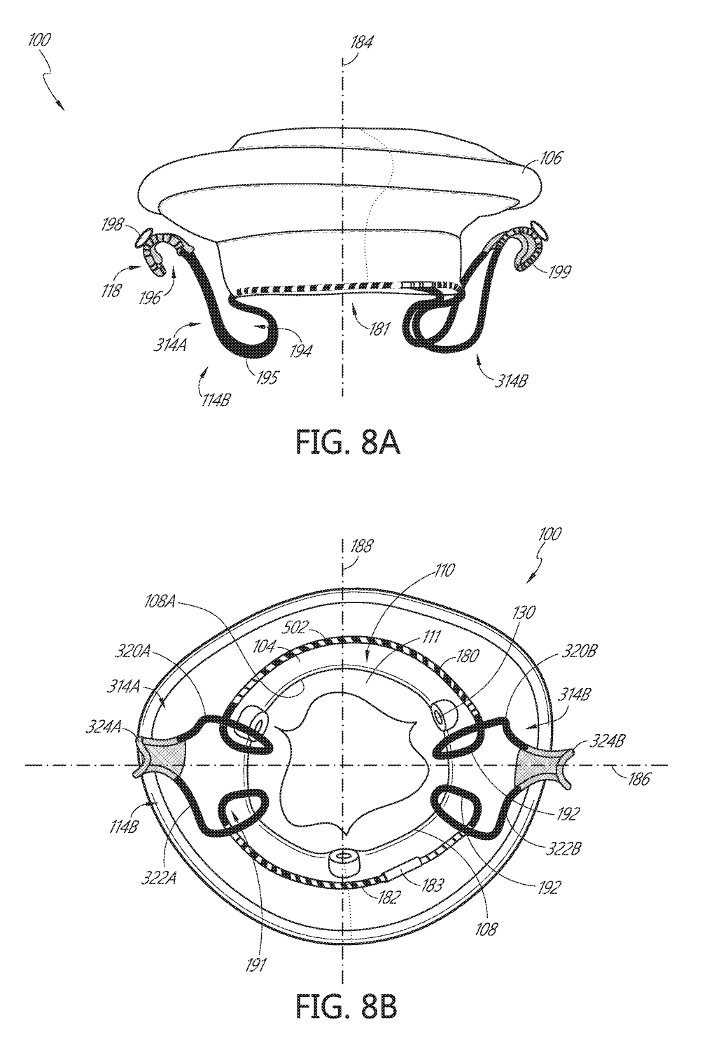

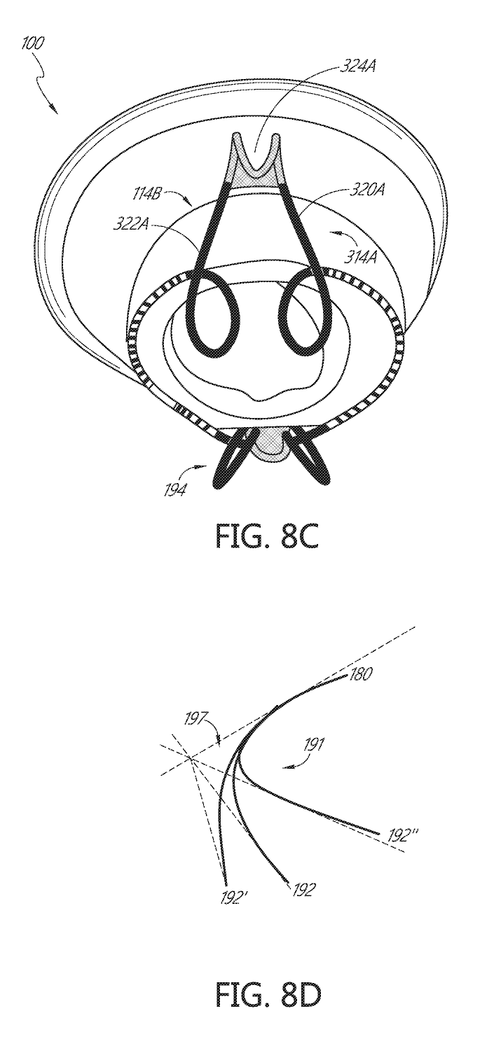

FIGS. 8A-C show the implant 100 described above with another embodiment of an anchor 114B that comprises a first and second anchor 314A, 314B. The features of the anchor 314A, 314B can be combined or included with any of the embodiments of the implant 100 discussed herein. As described above, the implant 100 can include the cuff or body 108 that extends between the inflow and outflow rings 102, 104. The cuff 108 can be adapted to support the valve 110 that is coupled to the cuff 108. The cuff 108 can be tubular with an inflow end and an outflow end corresponding to the inflow ring 102, outflow ring 104. The inner surface 108a of the cuff 108 can define a flow path through which blood can flow through the implant 100. The valve 110 can include one or more leaflets 111 positioned in the flow path defined by the inner surface 108a of the cuff 108. In some embodiments, the valve 110 is a tissue valve comprising one or more leaflets 111 that can be stitched or otherwise coupled at their ends to the cuff 108. Additional details, modified embodiments and components of the implant 100 shown in FIGS. 8A-C can be found in the description herein and the accompanying figures. The anchors 314A, 314B while described in the context of the implant 100 can also find utility with other configurations and modified embodiments of the implant 100 including embodiments in which the implants includes a stent based support structure and/or does not include an inflatable support structure.

In the illustrated embodiment, the implant 100 can include a first anchor 314A and a second anchor 314B that are connected to one another by a hoop structure 181, which, in the illustrated arrangement, comprises a first hoop structure 180 and a second hoop structure 182, as shown in FIG. 8B. In the illustrated embodiment, the first and second anchors 314A, 314B and the first and second hoop structures 180, 182 can be formed by a single piece of flexible material. In one embodiment, the first and second anchors 314A, 314B and the first and second hoop structures 180, 182 are made of a metal or metal alloy and in one embodiment the first and second anchors 314A, 314B and the first and second hoop structures 180, 182 are made of a shape memory alloy or a super elastic alloy such as nitinol. In some configurations, the anchors 314A, 314B and/or the hoop structures 180, 182 are made of a high strength, low modulus metal, such as, for example, a titanium alloy. The anchors 314A, 314B and/or the hoop structures 180, 182 can be made of wire having a diameter of 0.030 inch. In some arrangements, the anchors 314A, 314B and/or the hoop structures 180, 182 can be made of wire having a diameter that is in a range of about 0.005 inch to 0.080 inch. In certain configurations, the anchors 314A, 314B and/or the hoop structures 180, 182 can be made of wire having a diameter that is in a range of about 0.010 inch to 0.050 inch.

The anchors 314A, 314B and the first and second loop structures 180, 182 need not be formed with a single piece of material and need not be joined all together. In some embodiments, the anchors 314A, 314B and the first and second hoop structures 180, 182 are formed by a single wire that has its opposing ends crimped together by, for example, inserting opposing ends within a crimp tube 183 that is crimped. In other embodiments, the opposing ends can be welded or otherwise coupled to each other. In some configurations, the anchors 314A, 314B and the first and second hoop structures 180, 182 are a unitary structure that is laser cut from a tube. In one particular embodiment, the first and second anchors 314A, 314B and the first and second hoop structures 180, 182 are formed from a single wire that has its ends crimped together to form the hoop structure by, for example, inserting opposing ends within a crimp tube 183 that is crimped. In one particular embodiment, the first and second anchors 314A, 314B and the first and second hoop structures 180, 182 are formed from a single wire made of shape memory alloy or metal alloy such as nitinol that has its ends crimped together to form the hoop structure. In certain embodiments, the wire can be heat set into the configuration shown in FIGS. 8A-C.

In some configurations, the first and second hoop structures 180, 182 can be attached to the implant 100 in the vicinity of the outflow ring 104. In the illustrate embodiment, the hoop structures 180, 182 can be coupled to the implant 100 using sutures or stitching 502 In the illustrated embodiment, the first and second hoop structures 180, 182 when viewed together can have an ellipsoid shape, an oval shape, or an arched shape. In some configurations, the first and second hoop structures 180, 182 can have a shape that defines at least a portion of the circumference of a circle or oval. In the illustrated embodiment, the implant 100 has the two anchors 314A, 314B circumferentially spaced apart 180° from one another. In some configurations, the implant 100 can include one, three, or more than three anchors 314A, 314B. The anchors 314B can be non-uniformly distributed about the circumference of the implant 100. The anchors 314A, 314B can have identical shapes. In some configurations, the anchors 314A, 314B can have different shapes. For example, the anchor 314A that aligns near the left trigone 40 (shown in FIG. 2) can have a more elongate profile or a less elongate profile compared with the anchor 314B that aligns near the right trigone 42 (shown in FIG. 2). The shape of the anchors 314A, 314B can be tailored according to the anatomy of the mitral valve annulus onto which the implant 100 is seated. In the illustrated embodiment, the anchors 314A, 314B have a slightly different appearance. For example, the far wire of the right anchor 314B is more visible compared to the far wire of the left anchor 314A. In other words, the near wire of the left anchor 314A obscures the view of the far wire of the left anchor 314A more than the near wire of the right anchor 314A obscures the view of the far wire of the right anchor 314B. This can be because the left and right anchors 314A, 314B are not spaced apart circumferentially by exactly 1800 and because the left and right anchors 314A, 314B can have slightly different shapes.

For the sake of clarity, a coordinate system will be defined to simplify description of the anchors 314A, 314B and the hoop structures 180, 182. As shown in FIG. 8A, a longitudinal axis 184 can be aligned along the longitudinal axis of the implant 100, which can generally correspond to the direction of blood flow through the implant 100. Referring to FIG. 8B, a medial axis 186 can be aligned in the plane of the outflow ring 104 and can be interposed between the first and second hoop structures 180, 182. A transverse axis 188 can be aligned in the plane of the outflow ring 104 and can be oriented perpendicular to the medial axis 186, as shown in FIG. 8B. The medial axis 186 and the transverse axis 188 can both lie in a plane that is perpendicular to the longitudinal axis 184. This plane can be referred to as “the outflow plane.”

With continued reference to FIG. 8B, the anchors 314A, 314B can include a first bend 191 that can be located at an end of the first and/or second hoop structures 180, 182. The first bend 191 can curl inward from the periphery of the outflow ring 104 and toward the longitudinal axis 184. The first bend 191 can transition to an extension 192 that extends toward the longitudinal axis 184, as illustrated in FIG. 8B. In the illustrated embodiment, the extension 192 is located at roughly the same radial distance as the connection ports 130 that are used to connect the implant 100 with the PFL tubing 132 of the delivery catheter 200, as described above. In some configurations, the first bend 191 and the extension 192 can lie substantially within the outflow plane. However, in some embodiments, at least a portion of the first bend 191 and/or the extension 192 can extend outside of the outflow plane.

The anchor 314A, 314B can include a second bend 194 that connects the anchor 314A, 314B with the extension 192. Referring to FIGS. 8A and 8B, at least a portion of the second bend 194 can extend radially inward of the inner periphery of the outflow ring 104. In some configurations, at least a portion of the anchor 314A, 314B is disposed within the flow path (e.g., annulus of the valve 110). Accordingly, as located in FIGS. 8A and 8B, in the final or resting position at least a portion of the anchor 314A, 314B is positioned inwardly with respect to the outflow ring 104 of the implant 100. As shown in FIG. 8C, the second bend 194 can be oblique to the outflow plane. In some embodiments, the second bend 194 can be adapted so that the second bend 194 is substantially perpendicular to the outflow plane. For example, the second bend 194 can be formed so that the distal-most portion 195 (shown in FIG. 8A) of the second bend 194 can be aligned vertically over the extension 192 (shown in FIG. 8B). Another way to view the anchor 314A, 314B is that the anchor 314A, 314B can include a pair of anchors 314A, 314B that each includes a pair of bends that extend inwardly from the hoop structure 181 (shown in FIG. 8A) and then turn downwardly from the hoop structure 181 and then turn outwardly away from hoop structure 181 and then turn in an upward direction such that the tip portion 118 of each anchor 314A, 314B is positioned above the hoop structure 181 and outwardly respect to the hoop structure 181.

The anchor 314A, 314B can include a third bend 196 that is interposed between the second bend 194 and the tip portion 118 of the anchor 314A, 314B, as indicated in FIG. 8A. In the illustrated embodiment, the third bend 196 can have a curvature that is opposite to the curvature of the second bend 194, giving the anchor 314A, 314B an S-shape. As discussed in more detail below, the tip portion 118 can include a pad 198. The pad 198 can be disposed on an apical aspect of the anchor 314A, 314B, as shown in FIG. 8A. The pad 198 can be made of a compliant material (e.g., silicone). In some configurations, the pad 198 is adapted to reduce trauma caused by the anchor 314A, 314B to surrounding tissue.

The anchor 314A, 314B can include a cover 199. The cover 199 can cover a portion of the anchor 314A, 314B. In the illustrated embodiment, the cover 199 covers the anchor 314A, 314B near the tip portion 118 of the anchor 314A, 314B but does not cover the anchor 314A, 314B near the base portion 141 of the anchor 314A, 314B. As discussed in more detail below, the cover 199 can be adapted to avoid entrapping tissue (e.g., chordea tendineae) in the anchor 314A, 314B as the anchor 314A, 314B is deployed to secure the implant 100 in situ. In some configurations, the base portion 141 of the anchor 314A, 314B can be uncovered by the cover 199 in order to avoid blood stasis between the base portion 141 of the anchor 314A, 314B and the surrounding tissue.

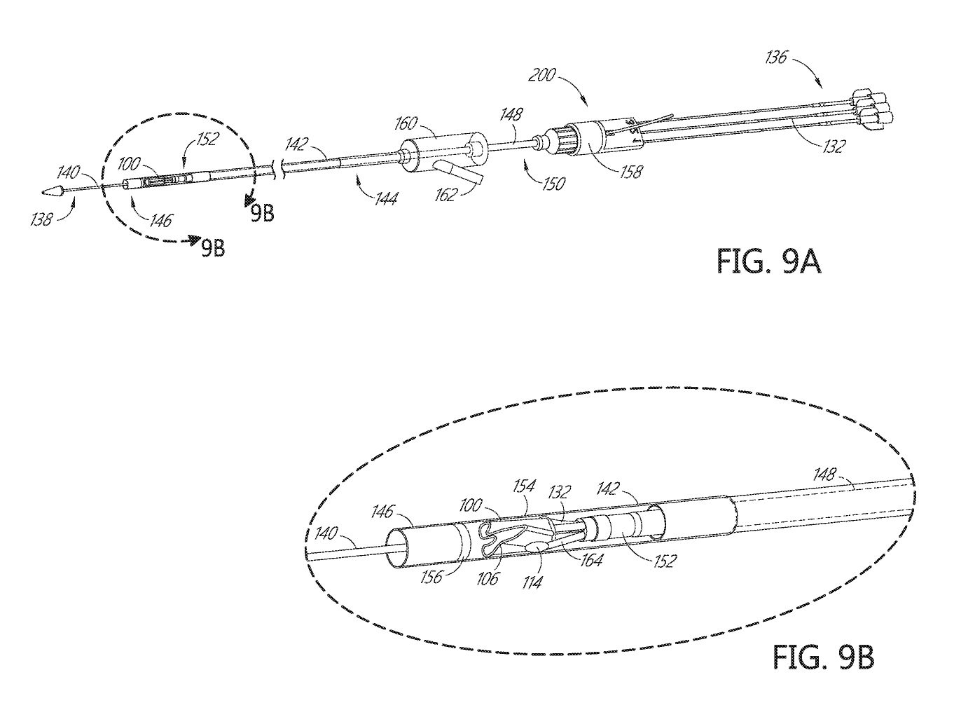

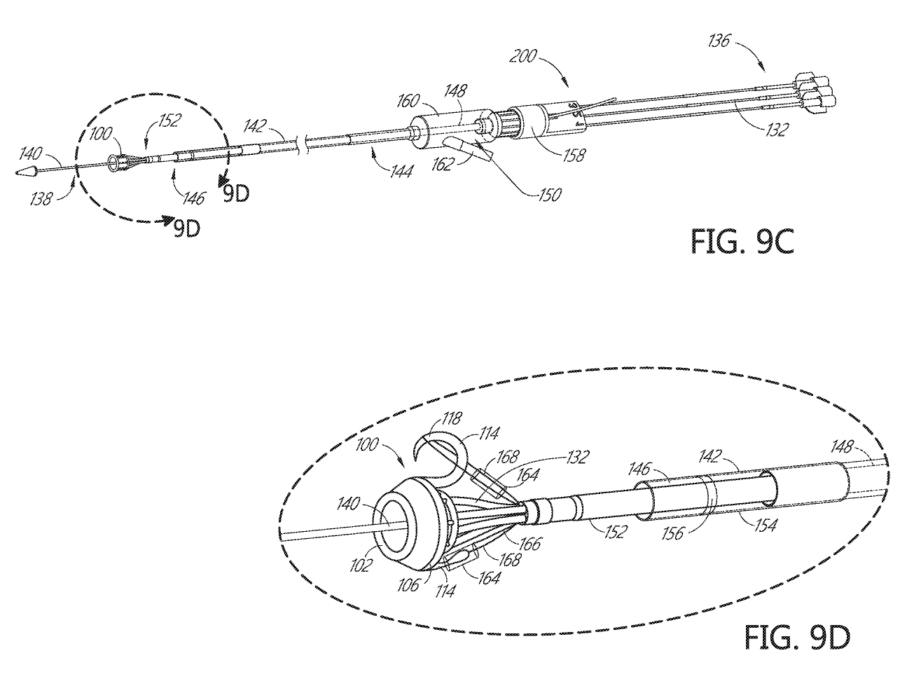

The first and second hoop structures 180, 182 can be adapted to better distribute stresses that are imposed on the implant 100 as the anchor 314A, 314B is moved into an extended position by moving the tip portion 118 away from atrial ring 106. In some embodiments, the profile of the implant 100 can be reduced by moving the anchor 314A, 314B into the extended position. As discussed in more detail below, the anchor 314A, 314B can be moved into the extended configuration in order to load the implant 100 into a delivery catheter (shown in FIG. 9A). In some embodiments, the anchor 314A, 314B can be moved into an extended configuration by applying to the tip portion 118 a force that is directed away from the apical ring 106.

As the tip portion 118 moves away from the atrial ring 106, the second bend 194 opens up, generating a counteracting force on the extension 192. The counteracting force that is imposed on the extension 192 generates a torque in the first and second hoop structures 180, 182. The torque tends to twist the first and second hoop structures 180, 182. As can be understood from FIG. 8B, when the anchor 314A, 314B are moved toward the extended position, the torque imposed on the first hoop structure 180 by the first anchor 314A will be offset by the torque imposed on the first hoop structure 180 by the second anchor 314B. Thus, the anchors 314A, 314B tend to twist either end of the first hoop structure 180 in opposite direction so that no net twist is imposed on the first hoop structure 180. In this way, the first and second anchors 314A, 314B help stabilize the extensions 192 on either end of the first hoop structure 180 and can help maintain the extensions 192 in the outflow plane. Accordingly, the outflow ring 104 can maintain a substantially circular shape or open configuration as the anchors 314A, 314B are moved into the extended position. The hoop structures 180, 182 can help maintain the flow path through the valve 110 and can help avoid having the flow path through the valve 110 from becoming substantially reduced when the anchors 314A, 314B are in the extended configuration.

FIG. 8D shows that the first bend 191 can have a bend angle 197 that characterizes the angle between the hoop structure 180 and the extension 192. In some configurations, the bend angle 197 can be less than 90°. In some embodiments, the bend angle 197 can be greater than 90°. The bend angle 197 of the embodiment shown in FIG. 8B is approximately 900. A bend angle of 90° provides the maximum torsion and load spread as the anchor 314A, 314B is moved to the extended configuration because the moment arm of the extension 192 is maximized by a bend angle 197 of 90°. Decreasing the bend angle 197 to less than 90° (see, e.g., extension 192″) decreases the torsion and load spread on the hoop structure 180 but tends to stabilize lateral movement of the anchor 314A, 314B, where lateral movement is a movement of the tip portion 118 of the anchor 314A, 314B away from the medial axis 186. Lateral movement of the anchor 314A, 314B can be undesirable as it can cause the implant 100 to shift (e.g., rotate about the longitudinal axis 184) relative to an intended position of the implant 100.

Referring again to the embodiment shown in FIG. 8B, the anchors 314A, 314B can be spaced apart circumferentially by 180° and in certain embodiments within 10 degrees of being apart circumferentially by 180° and in certain embodiments within 20 degrees of being apart circumferentially by 180°, which may reduce the ability of the implant 100 to be resist rotation about the medial axis 186. In many embodiments, the atrial ring 106 can stabilize the implant 100 and can reduce the tendency of the implant 100 to rotate about the medial axis 186. As discussed, the atrial ring 106 can be firmly seated onto the atrial aspect of the mitral valve annulus when the implant 100 is implanted in situ. In some embodiments, the implant 100 can include more than two anchors 314A, 314B (e.g., three anchors 314A) that are spaced apart circumferentially to increase the ability of the implant 100 to resist rotation about the medial axis 186. As shown in FIG. 8B, the implant 100 can include a tubular cuff 108 having an inner surface 108a that defines a pathway for blood flow, as discussed above. The valve 110 can be positioned within the pathway and coupled to the tubular cuff 108. The valve 110 can include one or more leaflets 111, as described above. The one or more leaflets 111 can be attached to the inner surface 108a of the cuff 108. The one or more leaflets 111 can be configured to permit flow in a first axial direction through the implant 100 and to inhibit flow in a second axial direction opposite to the first axial direction. The anchor 314A, 314B can be attached to the tubular cuff 108 for example by stitching and or sutures 502 as noted above. The anchor 314A, 314B can include a bend having a shape such that when the valve 110 is viewed in the second axial direction at least a portion of the bend extends radially inward of the inner surface 108a of the cuff.

In the embodiments of FIGS. 8A-8D, the anchors 314A, 314B are shown in combination with the embodiments implant 100 described and illustrated herein. However, it should be appreciated that certain embodiments, the anchors 314A, 314B can be used with an implant of a different configuration or type. For example, the anchors 314A, 314B may be used in combination with other implants such an implants that utilize a stent type support structure.

As mentioned, the inflow ring 102 and the outflow ring 104 can be inflated independently from one another and from the atrial ring 106. The separate inflation is useful during the positioning of the implant 100 at the implantation site. In some embodiments, the atrial ring 106 can be inflated before inflation of the inflow and outflow rings 102, 104 to seat the implant 100 before inflating the valve 110. In some variants, the inflow and outflow rings 102, 104 can be inflated before inflation of the atrial ring 106 so that blood can flow through the valve 110 while the implant 100 is positioned on the annulus of the native mitral valve 26.

During delivery, the cuff 108 and skirt 112 are limp and flexible providing a compact shape to fit inside a delivery sheath (shown in FIG. 9B). The cuff 108 and skirt 112 are therefore preferably made form a thin, flexible material that is biocompatible and may aid in tissue growth at the interface with the native tissue. A few examples of material may be Dacron, ePTFE, PTFE, TFE, woven material such as stainless steel, platinum, MP35N, polyester or other implantable metal or polymer. As mentioned above with reference to FIG. 4A, the implant 100 may have a tubular or funnel shape to allow for the native valve to be excluded beneath the wall of the skirt 112. Within the cuff 108 and the skirt 112, the inflation channels 117 can be connected to a catheter lumen (e.g., PFL tubing 132) for the delivery of an inflation media to define and add structure to the implant 100. The valve 110, which is configured such that a fluid, such as blood, may be allowed to flow in a single direction or limit flow in one or both directions, is positioned within the cuff 108. The attachment method of the valve 110 to the cuff 108 can be by conventional sewing, gluing, welding, interference or other means generally accepted by industry.

In one embodiment, the cuff 108 can have a diameter of between about 15 mm and about 30 mm and a length of between about 6 mm and about 70 mm. The wall thickness can have a range from about 0.01 mm to about 2 mm. In some variants, the cuff 108 may gain longitudinal support in situ from members formed by inflation channels 117 or formed by polymer or solid structural elements providing axial separation. The inner diameter of the cuff 108 may have a fixed dimension providing a constant size for valve attachment and a predictable valve open and closure function. Portions of the outer surface of the cuff 108 may optionally be compliant and allow the implant 100 to achieve interference fit with the native anatomy.

When inflated, the inflatable rings 102, 104, 106 can provide structural support to the inflatable implant 100 and/or help to secure the implant 100 in the heart 10. Uninflated, the implant 100 is a generally thin, flexible shapeless assembly that is preferably incapable of support and is advantageously able to take a small, reduced profile form in which it can be percutaneously inserted into the body. As will be explained in more detail below, in modified embodiments, the inflatable implant 100 may comprise any of a variety of configurations of inflation channels 117 that can be formed from other inflatable members in addition to or in the alternative to the inflation channels 117 shown in FIG. 5B. In one embodiment, the valve 110 has an expanded diameter that is greater than or equal to 22 millimeters and a maximum compressed diameter that is less than or equal to 6 millimeters (18F).

### Delivery Device

FIGS. 9A-9D illustrate an exemplary embodiment of a low crossing profile delivery catheter 200 that can be used to deliver the implant 100. In general, the delivery system comprises a delivery catheter 200, and the delivery catheter 200 comprises an elongate, flexible catheter body having a proximal end 136 and a distal end 138. In some variants, the delivery catheter 200 is configured for trans-apical delivery of the implant 100 to the heart 10. The catheter body can have an outer diameter of about 40 French or less particularly at the distal portion of the catheter body (i.e. the deployment portion). In certain embodiments, the delivery catheter 200 is configured for intravascular delivery of the implant 100 to the heart 10. The catheter body can have an outer diameter of about 18 French or less particularly at the distal portion of the catheter body (i.e. the deployment portion).

In some embodiments, the delivery catheter 200 also comprises a cardiovascular prosthetic implant 100 such as described herein at the distal end of the catheter body. As described herein, certain features of the implant 100 and delivery catheter 200 are particularly advantageous for facilitating delivering the cardiovascular prosthetic implant 100 within a catheter body having outer diameter of about 18, 22, 26 French or less while still maintaining a tissue valve thickness equal to or greater than about 0.011 inches and/or having an effective orifice area equal to or greater than about 1 cm squared, or in another embodiment, 1.3 cm squared or in another embodiment 1.5 cm squared. In such embodiments, the implant 100 may also have an expanded maximum diameter that is greater than or equal to about 22 mm. In some embodiments, at least one link exists between the catheter body and the implant 100. In some embodiments, the at least one link is the PFL tubing 132. In one embodiment, the delivery system is compatible with a guidewire 140 (e.g., 0.035″ or 0.038″ guidewire).

The implant 100 of certain embodiments of the present disclosure can include features that allow the implant 100 to be delivered by a low-profile delivery catheter 200. For example, the implant 100 can be inflatable, allowing the implant 100, when deflated, to be compactly folded and stowed within the delivery catheter 200. In addition, the implant 100 can include anchors 114 that can be attached to the implant 100 without requiring a circumferential support scaffold such as a stent structure made of metal or a plastic such as those that are often used to secure nitinol anchors to an expandable stent. In addition, the anchors 114 of the present disclosure can be forced into a straight configuration that reduces the profile of the anchor 114. The base portion 141 of the anchor 114 can be attached to the flexible skirt 112 of the implant 100 rather than to a rigid scaffold, thereby allowing the anchor 114 to be aligned within folds of the implant 100 and reducing the profile of the implant 100 when the implant 100 is deflated and stowed within the delivery catheter 200. In certain arrangements, when the implant 100 is positioned within the delivery catheter 200, the anchors 114 do not overlap with a circumferential support scaffold such as a stent based structure made of a metal or plastic in the constrained position within the delivery catheter 200. In certain arrangements, the anchors 114 are the only rigid or metallic components of the implant 100 while the implant 100 is positioned within the delivery catheter 100.

In general, the delivery catheter 200 can be constructed with extruded tubing using well known techniques in the industry. In some embodiments, the catheter 200 can incorporate braided or coiled wires and or ribbons into the tubing for providing stiffness and rotational torqueability. Stiffening wires may number between 1 and 64. In some embodiments, a braided configuration can be used that comprises between 8 and 32 wires or ribbon. If wires are used in other embodiments, the diameter can range from about 0.0005 inches to about 0.0070 inches. If a ribbon is used, the thickness is preferably less than the width, and ribbon thicknesses may range from about 0.0005 inches to about 0.0070 inches while the widths may range from about 0.0010 inches to about 0.0100 inches. In another embodiment, a coil is used as a stiffening member. The coil can comprise between 1 and 8 wires or ribbons that are wrapped around the circumference of the tube and embedded into the tube. The wires may be wound so that they are parallel to one another and in the curved plane of the surface of the tube, or multiple wires may be wrapped in opposing directions in separate layers. The dimensions of the wires or ribbons used for a coil can be similar to the dimensions used for a braid.

With reference to FIGS. 9A-9C, the catheter 200 comprises an outer tubular member 142 having a proximal end 144 and a distal end 146, and an inner tubular member 148 also having a proximal end 150 and a distal end 152. The inner tubular member 148 extends generally through the outer tubular member 142. The proximal end 150 of the inner tubular member 148 can extend generally past the proximal end 144 of the outer tubular member 142. The inner tubular member 148 can move longitudinally with respect to the outer tubular member 142. In some embodiments, the inner tubular member 148 is moved distally relative to the outer tubular member 142 to deploy the implant 100. In some variants, when the implant 100 is retracted within the delivery catheter 200, the distal end 152 of the inner tubular member 148 can be proximal to the distal end 146 of the outer tubular member 142, thereby constraining the implant 100 within the outer tubular member 142, as shown in FIG. 7B. When the implant 100 is deployed from the delivery catheter 200, the distal end 152 of the inner tubular member 148 can be distal to the distal end 146 of the outer tubular member 142, as shown in FIG. 9D.

The distal end 146 of the outer tubular member 142 can comprise a sheath jacket 154. In some embodiments, the sheath jacket 154 may comprise KYNAR tubing. The sheath jacket 154 can house the implant 100 in a retracted state for delivery to the implantation site. In some embodiments, the sheath jacket 154 is capable of transmitting at least a portion of light in the visible spectrum. This allows the orientation of the implant 100 to be visualized within the catheter 200. In some embodiments, an outer sheath marking band 156 may be located at the distal end 146 of the outer tubular member 142. The proximal end 150 of the inner tubular member 148 can be connected to a handle 158 for grasping and moving the inner tubular member 148 with respect to the outer tubular member 142. The proximal end 144 of the outer tubular member 142 can be connected to an outer sheath handle 160 for grasping and holding the outer tubular member 142 stationary with respect to the inner tubular member 148. A hemostasis seal (not shown) is preferably provided between the inner and outer tubular members 148, 142, and the hemostasis seal can be disposed in outer sheath handle 160. In some embodiments, the outer sheath handle 160 can comprise a sideport valve 162, and fluid can be passed into the outer tubular member through it.

Referring to FIG. 9B, the implant 100 can be configured to compactly store within the outer tubular member 142. In some embodiments, the rings 102, 104, 106 can be configured to fold over one another in a nesting fashion. The atrial ring 106 can be folded such that the crease of the atrial ring 106 is offset relative to the anchors 114, thereby reducing the profile of the folded implant 100. The anchors 114 can be contained within anchor sheaths 164. The anchor sheaths 164 can be offset from one another longitudinally or circumferentially so as to reduce the profile of the implant 100 when the implant 100 is stored inside the delivery catheter 200. In some embodiments, the anchors 114 are not contained in anchor sheaths 164. The anchors 114 can be held in the low-profile, straight configuration by applying tension to the tip portion 118 of the anchor 114, such as by a suture attached to the tip portion 118. In this way, the anchors 114 can be held in a low-profile configuration without requiring that the anchors 114 be contained within an anchor sheath 164. The PFL tubes 132 can be offset circumferentially with respect to one another to reduce the profile of the delivery catheter 200. In some embodiments, the connection ports 134 (shown in FIG. 5A) can be positioned so that the connection ports 134 are offset longitudinally with respect to one another to reduce the profile of the delivery catheter 200.

Referring to FIGS. 9C and 9D, the implant 100 can be deployed from the delivery catheter 200 by moving the inner tubular member 148 distally relative to the outer tubular member 142, thereby moving the implant distally past the distal end 146 of the outer tubular member 142. As shown in FIG. 9D, the anchor 114 can be held in a substantially linear configuration when sheathed in the anchor sheath 164. The anchor 114 can have a curled configuration when the anchor 114 is released from the anchor sheath 164. In some embodiments, the anchor sheath 164 can be coupled to an anchor sheath lead 166 that extends proximally through the delivery catheter 200 to the proximal end 136 of the delivery catheter 200. A user can release the anchor 114 from the anchor sheath 164 by pulling the anchor sheath lead 166 in the proximal direction. In some embodiments, a suture 168 is secured to the tip portion 118 of the anchor 114 to allow the anchor 114 to be re-sheathed in the anchor sheath 164. For example, the suture 168 can be configured as a loop, passing through a hole in the tip portion 118 of the anchor 114 and running through a central hole of the anchor sheath 164 so that the anchor sheath 164 surrounds at least a portion of the suture 168. A user can re-sheath the anchor 114 by pulling on the suture 168, thereby bring the anchor 114 into a linear configuration and drawing the anchor 114 into the anchor sheath 164. In some variants, the anchor 114 can be re-sheathed if the initial deployment of the anchor 114 fails to adequately grab heart tissue (e.g., left trigone 40 or right trigone 42). In some embodiments, the delivery catheter 200 does not include an anchor sheath 164, and the anchors 114 are held in the straight configuration by applying tension to the tip portion 118 of the anchor 114. The anchor 114 can be deployed by reducing the tension in the suture 168 that is secured to the tip portion 118. The suture 168 can allow a gradual or more controlled deployment of the anchor 114. In some embodiments, the tension in the suture 168 is slowly reduced to allow the anchor 114 to slowly transition into the spiral configuration. If the anchor 114 position is unsatisfactory, the anchor 114 can be brought out of the spiral configuration by increasing the tension in the suture 168, and the implant 100 can be repositioned (e.g., rotated or moved axially). In this way, the anchor 114 can be deployed under control until the anchor 114 is adequately attached to the surrounding tissue. When the anchor 114 adequately grabs or attaches to tissue, the suture 168 can be removed from the anchor 114 by cutting the suture loop and pulling one end of the suture 168 to draw the other end of the suture 168 through the hole in the anchor 114.

### Method of Use

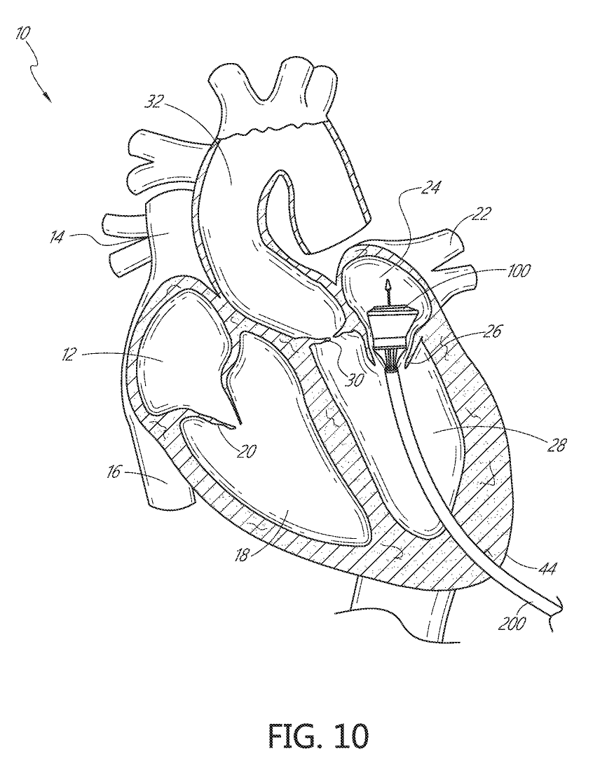

As discussed in greater detail herein, the implant 100 can be delivered to the mitral valve 26 by way of a trans-apical approach. The apical access site can be prepared according to standard practice. Referring to FIG. 10, the trans-apical procedure will be briefly described. A short incision (e.g., 3-4 inch long) can be made between two ribs to gain access to the apex 44 of the left ventricle 28. An incision is made through the apex 44 to gain access to the left ventricle 28. The delivery catheter 200 can then be introduced into the left ventricle 28 and advanced into the left atrium 24. The implant 100 can be deployed from the delivery catheter 200. As described in more detail below, the implant 100 can be inflated and seated within the annulus of the mitral valve 26. Flow through the implant 100 can be evaluated to confirm adequate placement of the implant 100. If needed, the implant 100 can be repositioned. In some variants, the implant can be deflated to reposition or recaptured the implant 100. In some embodiments, the implant 100 can be recaptured and removed from the heart 10. In this way, the implant 100 can be repositionable and retrievable. The surgeon can assess the outcome of the implant 100 before committing to the placement of the implant. These procedures will now be described in more detail.

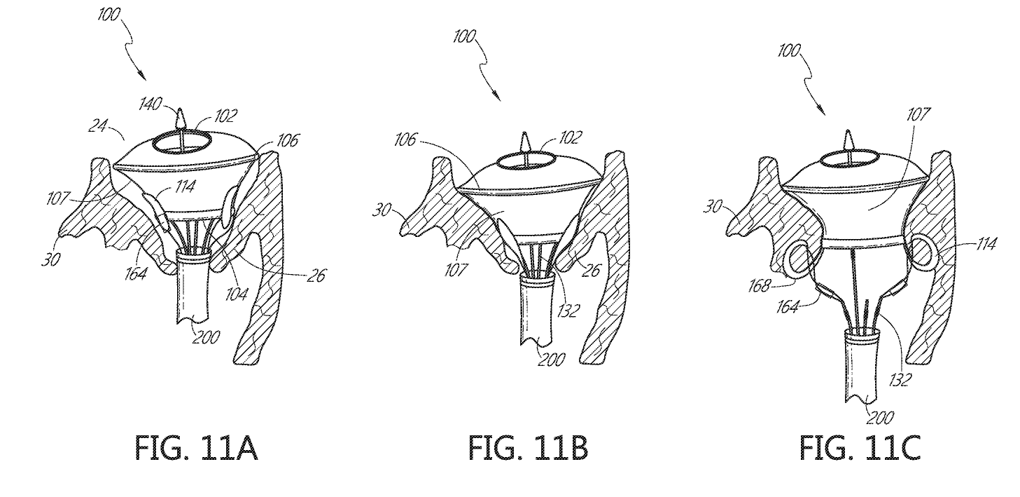

Referring to FIGS. 11A-11C, the catheter 200 carrying the implant 100 can be trans-apically advanced over a guidewire 140 to a position superior the native mitral valve 26. After the delivery catheter 200 is inserted over the guidewire 140 and advanced antegrade past the mitral valve 26 and into the left atrium 24, the implant 100 can be revealed or exposed by retracting the outer tubular member 142 partially or completely while holding the inner tubular member 148 stationary and allowing proper placement at or beneath the native valve 26. In some embodiments, the implant 100 may also be revealed by pushing the inner tubular member 148 distally while holding the outer tubular member 142 stationary. Once the implant 100 is unsheathed, it may be moved proximally or distally, and the fluid or inflation media may be introduced to the atrial ring 106 providing shape and structural integrity. In some embodiments, the inflow and outflow rings 102, 104 remain partially or completely deflated at this stage. In some embodiments, the entire inflatable structure 109 can be inflated or partially inflated. In some embodiments, the links are PRL tubes 132 can be used to both control the implant 100 and to fill the inflatable rings 102, 104, 106. In certain embodiments, the implant 100 is inflated initially with a with a non-solidifying inflation media (e.g., saline, gas). In some embodiments, the implant is inflated is inflated initially with a with a non-solidifying inflation media (e.g., saline, gas) and while inflated partially or fully the position of the implant 100 with respect to the native valve can be adjusted. For example, on one embodiment, the implant 100 can be proximally retracted after being fully or partially inflated to seat the atrial flange 196.

The deployment of the implant 100 can be controlled by the PFL tubes 132 that are detachably coupled to the implant 100. The PFL tubes 132 can be attached to the implant 100 at the connection points 134 described above. In some variants, the PFL tubes 132 can connect to the connection points 134 through a threaded coupling such as the couplings described above, thereby allowing the connection to withstand axial forces. In some embodiments, once the atrial ring 106 is inflated, for example, with a non-solidifying inflation media (e.g., saline, gas), the PFL tubes 132 can be used to pull back the implant 100 into or against the annulus of the mitral valve 26, as shown in FIG. 11B. The lower portion 107 of the skirt 112 of the implant 100 can be positioned to seal against the leaflets of the mitral valve 26. The anchors 114 can remain sheathed in the anchor sheaths 164 at this stage. The implant 100 can be rotated to align the anchors 114 with the left and right trigones 40, 42. For example, as shown in FIG. 2, the trigones 40, 42 are nearly aligned with the coaptation plane of the mitral valve leaflets. The implant 100 can be rotated to position the anchors 114 along the coaptation plane of the mitral valve 26 so that the anchors 114 do not interfere with the ability of the mitral valve leaflet to clear blood from the ventricle and reduce the risk of thrombosis, as described above.

Referring to FIG. 11C, the anchors 114 can be released from the anchor sheaths 164 by pulling the anchor sheaths 164 in the proximal direction. If the anchors 114 adequately attach to heart tissue (e.g., trigones) the securing suture 168 can be removed from the anchor 114. If the anchors 114 fail to adequately attach to heart tissue, the anchors 114 can be re-sheathed using the securing suture 168, as described above.

Once the implant 100 is securely seated in the annulus of the mitral valve 26, the inflow and outflow rings 102, 104 can be inflated to establish structural support to the valve 110. In some variants, the inflow and outflow rings 102, 104 are inflated for example, with non-solidifying inflation media (e.g., saline, gas), before releasing the anchors 114 or before pulling the atrial ring 106 onto or into the annulus of the mitral valve 26. In some embodiments, the implant 100 can be designed so that the sealing function of the atrial ring 106 is de-coupled from the valve-support function of the inflow and outflow rings 102, 104.

As discussed above, in some embodiments, the implant can be first inflated with non-solidifying inflation media (e.g., saline, gas). The non-solidifying inflation media can be displaced by a solidifying inflation media (e.g., epoxy) that can harden to form a more permanent support structure in vivo. Once the operator is satisfied with the position of the implant 100, the PFL tubes 132 are then disconnected, and the catheter 200 is withdrawn leaving the implant 100 behind (see FIG. 11C), along with the solidifying inflation media. The inflation media is allowed to solidify within the inflatable cuff. The disconnection method may include cutting the attachments, rotating screws, withdrawing or shearing pins, mechanically decoupling interlocked components, electrically separating a fuse joint, removing a trapped cylinder from a tube, fracturing a engineered zone, removing a colleting mechanism to expose a mechanical joint or many other techniques known in the industry. In modified embodiments, these steps may be reversed or their order modified if desired.

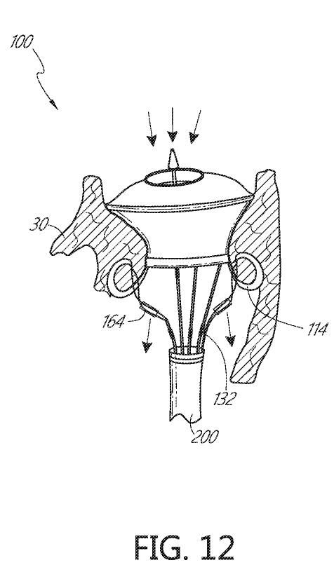

Referring to FIG. 12, the blood flow through the valve 110 of the implant 100 can be evaluated before disconnecting the implant 100 from the delivery catheter 200. In the illustrated embodiment, the flow through the valve 110 is being evaluated after the anchors 114 have been deployed from the anchor sheaths 164. In some embodiments, flow through the valve 110 can be evaluated before deploying the anchors 114 from the anchor sheaths 164. If the flow through the valve 110 is satisfactory, the PFL tubes 132 can be disconnected from the implant 100 and the delivery catheter 200 can be withdrawn from the heart 10, leaving the implant 100 behind. If the flow through the valve 110 is unsatisfactory, the implant 100 can be repositioned, as described above, and the flow through the valve 110 can be re-evaluated. In some embodiments, a contrast agent is delivered to a ventricular aspect of the posterior leaflet 33 of the mitral valve 26 (shown in FIG. 2) through a lumen of the delivery catheter 200. In some arrangements, a contrast agent is delivered to a ventricular aspect of the anterior leaflet 35 of the mitral valve 26 through a lumen of the delivery catheter 200. In some embodiments, a contrast agent is delivered through a lumen of the delivery catheter 200 to a ventricular aspect of both the posterior and anterior leaflets 33, 35 of the mitral valve 26.

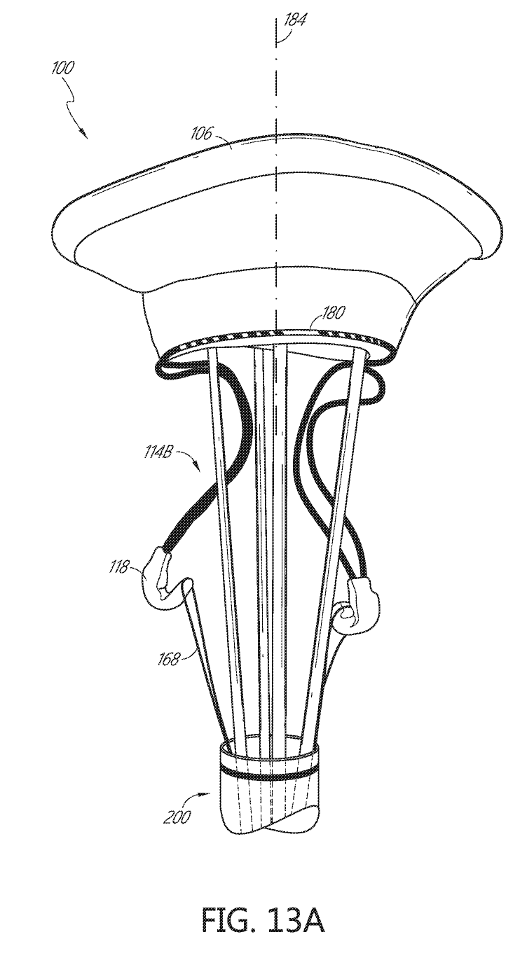

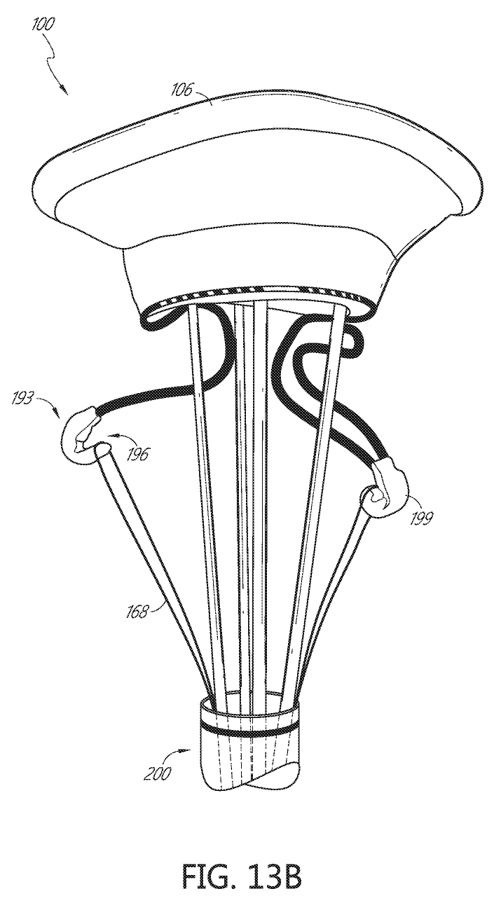

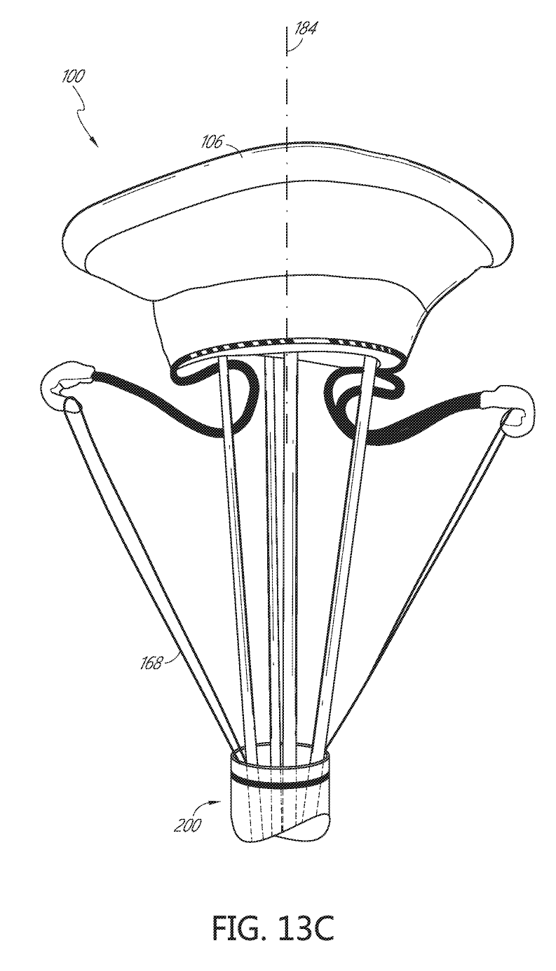

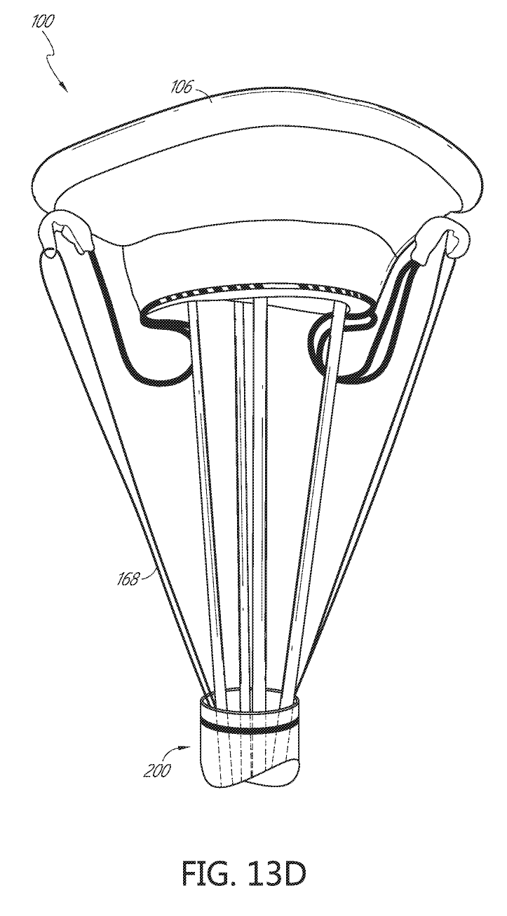

FIGS. 13A-D illustrate a method of deploying of an implant 100 having the anchors 314A, 314B of FIGS. 8A-D, which are labeled 114B in FIG. 13A, that are connected by the hoop structure 181 as described above. In FIG. 13A, the implant 100 has been deployed from the delivery catheter 200 and the atrial ring 106 has been inflated, as discussed above. The tip portion 118 can be held in the extended configuration by a suture 168 that is secured to the tip portion 118 of the anchor 314A, 314B. As mentioned, holding the tip portion 118 of the anchor 314A, 314B in the extended configuration can reduce the profile of the implant 100, allowing the implant 100 to be stored more compactly within the delivery catheter 200.

In some embodiments, the first and second hoop structures 180, 182 are collapsed toward the longitudinal axis 184 of the implant 100 in order to reduce the profile of the implant 100 for stowing the implant 100 within the delivery catheter 200. For example, the middle portion of the first hoop structure 180 can be pulled up (i.e., in the direction of the atrial ring 106) relative to the ends of the hoop structure 180 and pinched toward the longitudinal axis 184 to reduce the profile of the first hoop structure 180. The second hoop structure 182 (shown in FIG. 10B) can be similarly deformed and off-set from the first hoop structure 180 so that the first and second hoop structures 180, 182 nest over one another for storing within delivery catheter 200.

The tip portions 118 of the opposing anchors 314A, 314B can be similarly offset and nested to reduce the profile of the anchors 314A, 314B for storing within the delivery catheter 200. For example, in the configuration shown in FIGS. 8A-C, the first anchor 314A can have a first leg 320A and a second leg 322A that are joined by a first bridge 324A. The second anchor 314B can have a first leg 320B and a second leg 322B that are joined by a second bridge 324B. For compact storage in the delivery catheter 200, the wires of the anchors 314A, 314B can be offset and overlapped with one another so that a wire of each anchor 314A, 314B is interposed between the two wires of the other anchor 314A, 314B. For example, the first leg 320A of the first anchor 314A can be interposed between the first and second legs 320B, 322B of the second anchor 314B, thereby causing the second leg 322B of the second anchor 314B to be interposed between the first and second legs 320A, 322A of the first anchor 314A. The anchors 314A, 314B can have a notch or bend that is adapted to receive a portion of one of the wires of the opposing anchor 314A, 314B, thereby allowing the opposing anchor 314A, 314B to cross the other anchor 314A, 314B while maintaining a low profile. For example, the second bridge 324B can have a notch or bend that receives at least a portion of the first leg 320 of the first anchor 314A, thereby allowing the first anchor 314 to cross over the second anchor 314B while maintaining a compact configuration for storage inside a delivery catheter 200.

With continued reference to FIG. 13A, after the atrial ring 106 has been inflated the suture 168 can move distally out of the delivery catheter 200 to allow the tip portions 118 of the anchors 314A, 314B to move toward the atrial ring 106. In some configurations, the anchors 314A, 314B move simultaneously toward the atrial ring 106, as shown in FIGS. 13A-D. In some embodiments, one anchor 314A, 314B is held in the extended configuration while the other anchor 314A, 314B moves toward the atrial ring 106. In some configurations, at least a portion of the anchor 314A, 314B is longitudinally aligned with the flow path of the implant 100. As shown in FIG. 11A, the base portion 116 of the anchor 314A, 314B can comprise a pair of spaced apart wires that are uncovered, allowing blood to flow past the anchor 314A, 314B when the anchor 314A, 314B is aligned with the flow path of the implant 100. In some configurations, at least a portion of the anchor 314A, 314B remains in the flow path of the implant 100 as the anchor 314A, 314B moves from the extended configuration (FIG. 13A) to the deployed configuration (FIG. 13D).

Referring to FIG. 13B, the tip portion 118 of the anchor 314A, 314B can sweep away from the longitudinal axis 184 as the anchor 314A, 314B moves from the extended configuration toward the atrial ring 106. The anchor 314A, 314B can be configured so that the tip portion 118 traces a wide arc as the anchor 314A, 314B moves from the extended configuration to the deployed configuration. In some embodiments, the anchor 314A, 314B is sized so that the tip portion 118 slides along the ventricle wall as the anchor 314A, 314B moves from the extended configuration to the deployed configuration. As shown in the illustrated embodiment, at least a portion of the tip portion 118 can include a cover 199 that covers the spaced apart wires of the anchor 314A, 314B. The anchor 314A, 314B can be adapted so that the cover 199 pushes past tissue (e.g., chordea tendineae) as the anchor 314A, 314B moves into the deployed configuration, thereby avoiding entrapping tissue with the anchor 314A, 314B. As discussed above, the anchor 314A, 314B can include a third bend 196. The anchor 314A, 314B can be configured so that the third bend 196 creates a cam surface 193 that leads the tip portion 118 of the anchor 314A, 314B toward the atrial ring 106. The cam surface 193 can be adapted to slide past tissue that encounters the anchor 314A, 314B. For example, the cam surface 193 can be shaped to promote tissue sliding off of the tip portion 118 as the tip portion 118 moves toward the atrial ring 106. In some embodiments, the cam surface 193 and the cover 199 work together to push past tissue as the anchor 314A, 314B moves toward the atrial ring 106.

FIG. 13C illustrates a position of the anchor 314A, 314B when the tip portions 118 are a maximum distance away from the longitudinal axis 184 of the implant 100. As shown in FIG. 11C, in some configurations the tip portion 118 can extend radially beyond the atrial ring 106 as the anchor 314A, 314B moves from the extended configuration to the deployed configuration. In some embodiments, the tip portion 118 of the anchor 314A, 314B remains radially inward of the atrial ring 106 as the anchor 314A, 314B moves from the extended configuration to the deployed configuration.

FIG. 13D illustrates an embodiment of the anchor 314A, 314B in the deployed configuration. As discussed above, the anchor 314A, 314B can be configured to capture tissue (e.g., trigone) between the implant 100 and the anchor 314A, 314B when the anchor 314A, 314B is in the deployed configuration. In some embodiments, the anchor 314A, 314B can include a pad 198 (shown in FIG. 10A) to reduce trauma to the tissue that is captured by the anchor 314A, 314B and the implant 100. As mentioned, the pad 198 can be made of compliant material (e.g., silicone). In some embodiments, the pad 198 can be disposed at least partially on the cam surface 193 of the anchor 314A, 314B.

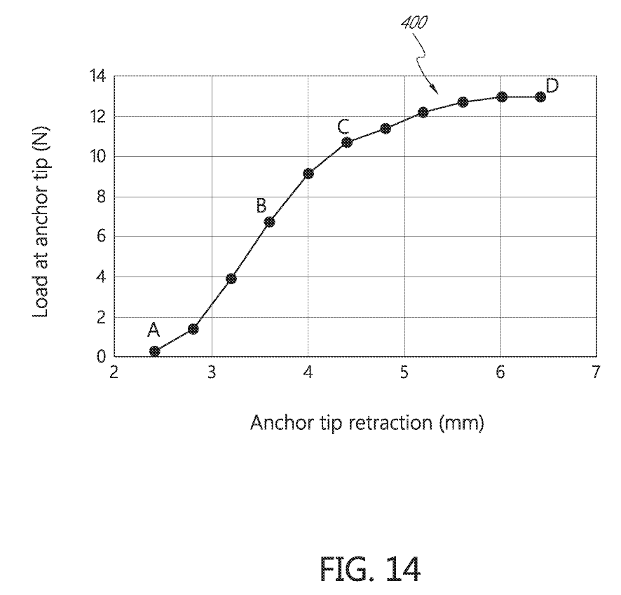

FIG. 14 illustrates a force curve 400 for an embodiment of the anchor 314A, 314B shown in FIGS. 11A-D. In other embodiments, the anchor 314A, 314B can have different or modified forced curves. The force curve 400 plots the force at the tip portion 118 of the anchor 314A, 314B as a function of the position of the anchor 314A, 314B from the extended configuration. The letters “A” through “D” on the curve 400 indicate the approximate position of the anchor 314A, 314B in the corresponding FIGS. 13A-D. As can be seen in FIG. 12, the anchor 314A, 314B exerts the maximum force (approximately 13 Newtons) when the anchor 314A, 314B is in the deployed configuration (position D). The anchor 314A, 314B exerts the minimum force (approximately 0.5 Newtons) when the anchor 314A, 314B is in the extended configuration (position A). The anchor 314A, 314B can be adapted to provide a different force curve 400. For example, the anchor 314A, 314B can be adapted to provide a larger or smaller force when the anchor 314A, 314B is in the deployed configuration (position D). The anchor 314A, 314B can also be adapted to provide a different shape to the force curve 400. For example, the anchor 314A, 314B can be adapted so that the force curve 400 has a maximum near “B” or “C” that declines as the anchor 314A, 314B moves toward “D”. In many embodiments, the force at the deployed position “D” is selected to be sufficient to embed the anchor 314A, 314B into surrounding tissue. The anchor 314A, 314B can include features (e.g., a pad 196, a cam surface 193) that reduce trauma to the tissue in which the anchor 314A, 314B embeds. In certain embodiments, the force at the deployed position “D” is selected to be sufficient to maintain throughout the cardiac cycle contact between the anchor 314A, 314B and the tissue in which the anchor 314A, 314B is embedded. For example, the anchor 314A, 314B can be adapted so that the anchor 314A, 314B exerts sufficient force in the deployed configuration to avoid the anchor 314A, 314B bouncing on and off of the tissue that is contacted by the anchor 314A, 314B when the anchor 314A, 314B is in the deployed configuration.

The above-described methods generally describe an embodiment for the replacement of the mitral valve 26. However, similar methods could be used to replace the pulmonary valve or the aortic valve or tricuspid valves. For example, the pulmonary valve could be accessed through the venous system, either through the femoral vein or the jugular vein. The aortic valve could be accessed through the venous system and then trans-septaly accessing the left atrium from the right atrium. Alternatively, the aortic valve could be accessed through the arterial system as described for the mitral valve, additionally the catheter 200 can be used to pass through the aortic valve 30 and then back up to the mitral valve 26. Additional description of mitral valve and pulmonary valve replacement in general can be found in U.S. Patent Publication No. 2009/0088836 to Bishop et al.

### Implant Recovery

Current valve systems are often deployed through a stent-based mechanism where the valve is sewn to the support structure. In the inflated embodiments described herein, the structure is added to the implant secondarily via the inflation fluid. This allows the user to inflate or pressurize the implant 100 with any number of media including one that will solidify. As such, if the operator desires, the implant 100 can be moved before the inflation media is solidified or depressurization can allow for movement of the implant 100 within the body. Since catheter-based devices tend to be small in diameter to reduce trauma to the vessel and allow for easier access to entry, it often difficult to remove devices such as stents once they have been exposed or introduced into the vasculature. However, as will be explained below, a device described herein enables a percutaneous prosthetic mitral valve to be recovered from the body and reintroduced retrograde to the introducer.

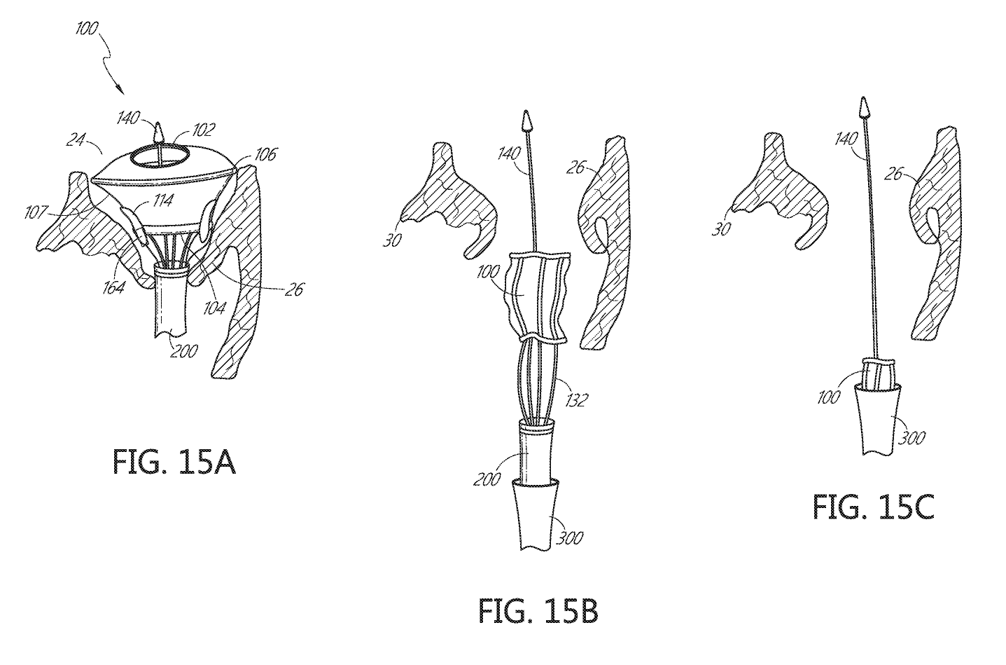

With reference to FIGS. 15A-C, the deployment control device also provides a method for retracting the implant 100 back into a recovery catheter 300 if the result is not satisfactory, or if the sizing of the implant could be optimized. Thus, after the implant 100 is fully or partially deployed (FIG. 15A), in addition to providing a mechanism to transmit axial force to the implant 100, the PFL tubes 132 described above provide a guide or ramp to pull the implant 100 back into the delivery catheter 200 or into the recovery catheter 300 as the implant 100 is retracted as shown in FIGS. 15B and 15C. In some embodiments, the outer tubular member 142 is retracted out of the heart 10 while leaving the inner tubular member 148 still attached to the implant 100 prior to introducing the recovery catheter 300.

To recapture an inflatable implant 100, the implant 100 is first deflated (FIG. 15B). In some embodiment, the implant 100 may be retracted to the tip of the inner tubular member 148 by pulling the PFL tubing 132 proximally, and the implant 100 and the delivery catheter 200 are then retracted to the tip of the recovery catheter 300. The inner sheath handle 158 (shown in FIG. 9A) may be removed by unthreading the distal portion and sliding off at the proximal end of the delivery catheter 200. In some embodiments, the connections on the proximal end of the PFL tubing 132 may be cut off for the removal of the inner sheath handle 158. Optionally a pushing tube can be loaded over the guidewire 140 and PFL tubing until adjacent to the proximal end of the inner tubular member 148. The outer tubular member 142 can then be removed from the delivery catheter 200, while keeping the implant 100 stationary.

The recovery catheter 300 can then be advanced over the guidewire 140 and the inner tubular member 148. Once the recovery catheter 300 is proximate to the implant 100, the recovery sheath is retracted to expose the basket section. The implant 100 can then be retracted into the basket section (FIG. 15C). Once the implant 100 is completely inside the basket section, in some embodiments, the PFL tubes 132 are adjusted to offset the connection points in the implant 100 to allow more compact fold. The recovery catheter 300 is then slowly pulled back through the introducer and out of the patient. In some embodiments, the recovery catheter 300 and/or delivery catheter 200 can include a plow-like element (not shown) that is configured to push the implant 100 back into the delivery catheter 200 and/or recovery catheter 300. For example, the recovery catheter 300 can include a plow-like element that tapers in the distal direction so that the element has a proximal face that is flared relative to the distal portion of the element. The element can be pushed distally past the inflated implant 100 and drawn proximally back after the implant 100 has been deflated, thereby allowing the flared proximal face to push the implant 100 in the proximal direction into the basket section of the recovery catheter.

In some configurations, the implant 100 is drawn into the recovery catheter 300 in a sideways orientation. As described above, before the implant 100 is fully released from the delivery catheter 200, a suture 168 can be attached to the anchor 114. In some methods of retrieving the implant 100 into a recovery catheter 300, the implant 100 is deflated while the anchors 114 remain in the deployed configuration (e.g., anchored to trigone tissue). The recovery catheter 300 can be advanced toward the implant 100 over the guidewire 140 and the sutures 168, as described above. One of the anchors 114 can be moved from the deployed configuration into the extended configuration while the other anchor 114 is left in the deployed configuration. For example, the suture 168 that is attached to a tip portion 118 of one of the anchors 114 can be used to pull the tip portion 118 away from the atrial ring 106 and into the extended configuration. As the suture 168 is used to pull the anchor 114 into the extended configuration, the implant 100 will pivot about the anchor 114 that is still deployed. The implant 100 can rotate about the deployed anchor 114 so that the side of the implant faces toward the distal opening of the recovery catheter 300. Accordingly, the central lumen of the implant 100 will be substantially transverse to the lumen of the recovery catheter 300. The delivery catheter 300 can be advanced toward the implant 100 to draw the implant 100 into the delivery catheter 300. Once the implant 100 is at least partially inside the recovery catheter 300, the anchor 114 that is still deployed can then be moved into the extended configuration, thereby completing detachment of the implant 100 from the tissue (e.g., trigones).

### CONCLUSION

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, the terms “approximately”, “about”, and “substantially” may refer to an amount that is within less than 10% of, within less than 5% of, within less than 1% of, within less than 0.1% of, and within less than 0.01% of the stated amount or characteristic. Numbers preceded by a term such as “about” or “approximately” also include the recited numbers. For example, “about 3.5 mm” includes “3.5 mm.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

1. A cardiovascular prosthetic valve implant, the valve comprising:
a cuff having an inner surface that defines a pathway for blood flow;
a valve positioned within the pathway and coupled to the cuff, the valve configured to permit flow in a first direction through the implant and to inhibit flow in a second direction opposite to the first axial direction;
an inflatable structure coupled to the cuff, the inflatable structure comprising at least an inflow ring, an outflow ring, and an atrial ring, the atrial ring having an outer diameter greater than the inflow and outflow rings.

2. The cardiovascular prosthetic valve implant of claim 1, wherein the cuff extends between the inflow ring and the outflow ring.

3. The cardiovascular prosthetic valve implant of claim 1, comprising a skirt that extends between the inflow ring, the atrial ring and the outflow ring.

4. The cardiovascular prosthetic valve implant of claim 3, wherein a space is defined between the skirt and the cuff.

5. The cardiovascular prosthetic valve implant of claim 4, wherein the skirt is formed of a material that permits blood to enter the space between the skirt and the cuff.

6. The cardiovascular prosthetic valve implant of claim 1, wherein the atrial ring has an ellipse shape.

7. The cardiovascular prosthetic valve implant of claim 6, wherein at least one of the inflow ring and the outflow ring is positioned off-center with respect to the atrial ring.

8. A cardiovascular prosthetic valve implant, the valve comprising:
a cuff having an inner surface that defines a pathway for blood flow, the cuff supported by an inflatable structure including at least one ring;
a valve positioned within the pathway and coupled to the cuff, the valve configured to permit flow in a first direction through the implant and to inhibit flow in a second axial direction opposite to the first direction; and
an atrial flange comprising an atrial ring and a skirt that extends between the ring of the cuff and the ring of the atrial flange.

9. The cardiovascular prosthetic valve implant of claim 8, wherein a space is defined between the skirt and the cuff.

10. The cardiovascular prosthetic valve implant of claim 8, wherein the skirt is formed of a material that permits blood to enter the space between the skirt and the cuff.

11. The cardiovascular prosthetic valve implant of claim 8, wherein the ring of the atrial flange has an ellipse shape.

12. The cardiovascular prosthetic valve implant of claim 11, wherein the ring of the cuff is positioned off-center with respect to the ring of the atrial flange.

13. A cardiovascular prosthetic valve implant, the valve comprising:
a tubular cuff having an inner surface that defines a pathway for blood flow, the tubular cuff comprising a first end having a first diameter and a second end having a second diameter;
a valve positioned within the pathway and coupled to the tubular cuff, the valve configured to permit flow in a first axial direction through the implant and to inhibit flow in a second axial direction opposite to the first axial direction; and
an atrial flange comprising an atrial ring having a diameter greater than the diameter first and second ends of the tubular cuff and a skirt that extends between the first end of the tubular cuff to the atrial ring and from the atrial ring to the second end of the tubular cuff to form a space between the skirt and the tubular cuff.

14. The cardiovascular prosthetic valve implant of claim 13, wherein the skirt is formed of a material that permits blood to enter the space between the skirt and the cuff.

15. The cardiovascular prosthetic valve implant of claim 13, wherein the atrial ring of the atrial flange has an ellipse shape.

16. The cardiovascular prosthetic valve implant of claim 13, wherein the tubular cuff is positioned off-center with respect to the ring of the atrial flange.

17. (canceled)

18. (canceled)

19. (canceled)

20. (canceled)

21. (canceled)

22. (canceled)

23. (canceled)

24. The cardiovascular prosthetic valve implant of claim 1, comprising a first anchor, a second anchor and a hoop structure, the first and second anchor each configured to move between an extended configuration and a deployed configuration, the hoop structure connecting the first anchor to the second anchor.

25. The cardiovascular prosthetic valve implant of claim 1, comprising a first anchor coupled to the outflow ring of the inflatable structure, the first anchor comprising a bend that extends at least partially radially inwardly into the pathway for blood flow.

26. The cardiovascular prosthetic valve implant of claim 8, comprising a first anchor, a second anchor and a hoop structure, the first and second anchor each configured to move between an extended configuration and a deployed configuration, the hoop structure connecting the first anchor to the second anchor.

27. The cardiovascular prosthetic valve implant of claim 8, comprising a first anchor coupled to the cuff, the first anchor comprising a bend that extends at least partially radially inwardly into the into the pathway for blood flow.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)
